(19) United States
(12) Patent Application Publication
(10) Pub. No.: US 2025/0025601 A1
(43) Pub. Date: Jan. 23, 2025

(54) PNEUMATOSPUN BIOPOLYMER SCAFFOLDS AND METHODS FOR THEIR PRODUCTION

(71) Applicant: SCRIPPS HEALTH, San Diego, CA (US)

(72) Inventors: Darryl D. D'LIMA, San Diego, CA (US); Erik W. DORTHE, San Diego, CA (US); Austin B. WILLIAMS, San Diego, CA (US); Shawn P. GROGAN, Mission Viejo, CA (US)

(21) Appl. No.: 18/709,430

(22) PCT Filed: Nov. 11, 2022
(86) PCT No.: PCT/US2022/049739

§ 371 Date: May 10, 2024

Related U.S. Application Data
(60) Provisional application No. 63/278,815, filed on Nov. 12, 2021.

(51) Int. Cl. A61L 27/26; A61L 27/38; C08L 5/10; C08L 89/06

(52) U.S. Cl. CPC A61L 27/26; A61L 27/3834; C08L 5/10; C08L 89/06; A61L 2430/06

(57) ABSTRACT

Disclosed herein are methods of producing a scaffold by producing a biopolymer scaffold composition by pneumatospinning a polymer solution comprising polymer fibers onto a collector; feeding the polymer fiber solution comprising the polymer fiber into an air brush; applying a gas pressure around the polymer fiber solution comprising the polymer fiber in the airbrush; ejecting the polymer fiber from the air brush onto the collector; crosslinking the polymer fibers; and seeding a plurality of cells to the polymer scaffold composition.

### CROSS-REFERENCE

This application claims the benefit of U.S. Provisional Application No. 63/278,815, filed Nov. 12, 2021, which is incorporated by reference herein in its entirety.

### BACKGROUND

Soft connective tissues such as tendons, ligaments, meniscus, and cartilage are prone to damage and injury. Various approaches have been taken to develop repairs to these types of connective tissues and the joints in which they reside. Surgical repair of cartilage and ligaments has been a common strategy for injury or damage that is unlikely to heal without medical intervention. Some long-standing methods of surgical repair of connective tissues have included the use of cadaver tissue or autografting as a supply for the replacement tissue. The use of each of these tissue types during replacement surgery has presented some associated problems.

Allografts, which are tissue grafts from a donor, have been demonstrated to increase scar formation as well as increase the likelihood of an immune response directed against the donor tissue. Allografts are also present in a limited supply which can create a bottleneck for availability of tissue required to conduct this type of surgical repair. Autografting, the grafting of a tissue from one point to another in the same individual's body, has been developed as an alternative to using donor tissue. Lower risk of both promoting an immune response and possible tissue rejection have been reasons to promote autograft tissue replacement surgery. This invasive type of surgery often comes with extended lengths of surgery as well as additional procedures to recover and prepare autologous tissue.

Factors inherent to both the extent of a soft tissue injury and the characteristics of the tissue at the injury site have been shown to affect the likelihood of successful surgical repairs. For instance, damage to a tissue site that is highly vascularized has a higher success rate for repairs than in injuries to avascular tissues. This has limited the options for certain repairs in avascular tissues. The step of tissue replacement can sometimes be bypassed in smaller tears in meniscus or fibrocartilage and soft connective tissues with sutures, screws or dart to bridge small gaps in damaged tissue. Large tears often necessitate removal of the loose tissue to prevent further damage to the joint and the risk of arthritic complications.

These factors have led to interest in developing suitable artificial scaffolds for implantation at locations of soft tissue damage. Scaffolds may be engineered to promote favorable cellular interactions which contribute to the formation of functional tissues at the site of implantation. Cells can be seeded onto scaffolds, which can then provide a framework for growing a three-dimensional tissue structure. Many characteristics prove important to the successful function of implanted artificial scaffolds. One of these characteristics is biomechanical flexibility. Two other important traits are strength and biodegradability. Scaffolds that are more porous have been considered desirable as they could allow more effective migration and integration of host cells during tissue healing. Lastly, biological compatibility is perhaps the most critical when considering scaffold material and design.

Both synthetic and natural materials have been utilized in the construction of biocompatible scaffolds. Some synthetic scaffolds have been created using silicone, polylactic acid (PLA), polyglycolic acid (PGA) or a combination of PLA and PGA to create poly-lactic-co-glycolic acid (PLGA). A scaffold that mimics extracellular matrix has been considered desirable in that this type of structure may more closely resemble the native microenvironments that allow mesenchymal cells to attach and migrate. This may also allow effective diffusion and retention of extracellular signaling molecules. Scaffolds have been fashioned out of protein-based materials such as collagen, fibrin and various glycosaminoglycans.

Various techniques have been described to synthesize a scaffold for tissue engineering. Textile technologies have been used to create non-woven meshes of various polymers. Solvent casting and particulate leaching (SCPL) can result in creation of scaffold structures with a regular porosity; however, this technique limits the thickness of the forms. Organic solvents used with SCPL must be fully removed to avoid potentially damaging cells seeded to the scaffold. This step can be time consuming and costly. Gas foaming is another technique which has been developed to overcome the need for organic solvents in the synthesis process; however, scaffolds formed by this method often lack an interconnected structure. Electrospinning is another scaffold synthesis method that can produce continuous fibers of varying diameters. Using this technique, scaffold material is first dissolved and placed in a syringe. This solution is fed through a needle, and voltage is then applied to the needle tip. A nearby conductive surface is used for collection. This application causes the solution to eject a fine stream toward the collection surface. The solvent then evaporates, leaving solid continuous fibers on the collection surface. Parameters such as the distance between the needle and collection surface, the voltage used, and the flow rate of the solution can all be varied to create suitable fiber morphology for scaffolds. One drawback to electrospinning is commercial scalability. Thick scaffolds produced by electrospinning are costly and time consuming.

### SUMMARY

Provided herein are methods comprising: applying a gas pressure around a polymer fiber solution comprising a polymer fiber to eject the polymer fiber solution; collecting the polymer fiber by a collector to form a scaffold, wherein the collector comprises a shape of a meniscus. In some embodiments, the collector comprises a flat convex shape, a concave shape, or a wave shape. In some embodiments, the collector comprises a first curvature and a second curvature on a surface of the collector, wherein a degree of curvature of the first curvature and a degree of curvature of the second curvature are different. In some embodiments, the scaffold comprises the shape of the meniscus. In some embodiments, scaffold comprises a shape of a human meniscus. In some embodiments, collector comprises a mesh collector or a solid collector. In some embodiments, collector rotates at least about 10 revolutions per minute (rpm). In some embodiments, the collector rotates at least about 600 rpm. In some embodiments, the collector rotates at a constant speed. In some embodiments, the collector rotates at a varying speed. In some embodiments, the collector comprises a rotating collector, a stationary collector, or a translating collector. In some embodiments, the gas pressure is at least about 1 pound per square inch (psi). In some embodiments, the gas pressure is at least about 10 psi. In some embodiments, the gas pressure is at least about 30 psi. In some embodiments, the gas pressure comprises air pressure. In some embodiments, the applying comprises applying the gas pressure around the polymer fiber solution using an air brush. In some embodiments, the polymer fiber solution moves into the air brush via an electromagnetic pressure, an electromechanical pressure, gravity, a hydraulic pressure, a mechanical extrusion pressure, a pneumatic pressure, or a combination thereof. In some embodiments, the polymer fiber solution moves into the air brush via the mechanical extrusion pressure using a syringe pump. In some embodiments, the air brush comprises an inner feed line. In some embodiments, the polymer fiber solution moves inside the inner feed line. In some embodiments, the polymer fiber solution moves inside the inner feed line continuously during the collecting. In some embodiments, the air brush comprises an outer air line. In some embodiments, the air moves inside the outer air line. In some embodiments, the air brush comprises a nozzle to eject the polymer fiber solution. In some embodiments, the air brush comprises at least two nozzles to eject the polymer fiber solution. In some embodiments, the nozzle is uniaxial, coaxial, triaxial. tetraaxial, or pentaaxial. In some embodiments, the nozzle is at least about 30 μm. In some embodiments, the nozzle is at least about 300 μm. In some embodiments, the collecting of comprises at most about 10 hours. In some embodiments, the collecting of comprises at most about 5 hours. In some embodiments, the polymer fiber comprises a carbohydrate glass, a carbon nanotubes, a chitosan, a collagen, a dextran, a fibrinogen, a fibroin, a gelatin, a glycosaminoglycan, a hydroxyapatite, a keratin, a latex, a nanobioactive glass, a poly(ester-urethane)urea, a poly(ethylene glycol), a poly(ethylene oxide, a poly(lactic acid-co-glycolic acid), a poly(lactide-co-caprolactone), a poly (propylene carbonate), a polyaniline, a polydiaxanone, a polyglycolic acid, a polylactic acid, or a combination thereof. In some embodiments, the collagen comprises a type I collagen or a type II collagen. In some embodiments, the collagen comprises a type III collagen. In some embodiments, the collagen comprises a type I collagen. In some embodiments, the collagen is a methacrylated collagen. In some embodiments, the polymer fiber comprises the glycosaminoglycan. In some embodiments, the method further comprises seeding the scaffold with a cell. In some embodiments, the cell comprises a chondrocyte, an endothelial cell, a meniscus cell, a mesenchymal cell, or a synovial cell. In some embodiments, the cell comprises a stem cell. In some embodiments, the stem cell comprises an Infrapatellar fat pad (IPFP)-derived stem cell. In some embodiments, the seeding of comprises a plurality of cells. In some embodiments, the plurality of cells comprises at least about 1×10\hat{ }3 cells. In some embodiments, the plurality of cells comprises at least about 5×10\hat{ }6 cells. In some embodiments, the scaffold is suitable for implantation. In some embodiments, the scaffold comprises a Young's modulus. In some embodiments, the Young's modulus is about 10 MPa to about 1000 MPa. In some embodiments, the Young's modulus is about 1 MPa to about 2000 MPa. In some embodiments, the method further comprises culturing the cell seeded in the scaffold. In some embodiments, the method further comprises producing a plurality of polymer scaffold. In some embodiments, the method further comprises combining the plurality of polymer scaffold compositions to form a multilayer construct

In another aspect, there are provided methods comprising applying a gas pressure around a polymer fiber solution comprising a polymer fiber to eject the polymer fiber solution; forming a scaffold comprising the polymer, wherein the scaffold has a thickness of at least about 300 micrometer (μm) and a porosity of at least about 40%. In some embodiments, the scaffold has a thickness of at least about 1 millimeter (mm). In some embodiments, the scaffold has a porosity of at least about 50%. In some embodiments, the porosity is measured by an average size of a plurality of pores of scanning electron microscopy (SEM) image of the scaffold. In some embodiments, the scaffold has a shape of a bone, a cartilage, a fibrous tissue, a muscle, or a combination thereof. In some embodiments, the fibrous tissue comprises a band, a blood vessel, a bursas, a labrum, a ligament, a meniscus, a tendon, an enthesis, or a combination thereof. In some embodiments, forming comprises forming the scaffold comprising the polymer with a collector. In some embodiments, the collector comprises a mesh collector or a solid collector. In some embodiments, the collector rotates at a constant speed. In some embodiments, the collector rotates at a varying speed. In some embodiments, the collector rotates at least about 10 revolutions per minute (rpm). In some embodiments, the collector rotates at least about 600 rpm. In some embodiments, the collector comprises a rotating collector, a stationary collector, or a translating collector. In some embodiments, the gas pressure is at least about 1 pound per square inch (psi). In some embodiments, the gas pressure is at least about 10 psi. In some embodiments, the gas pressure is at least about 30 psi. In some embodiments, the gas pressure is greater than 30 psi. In some embodiments, the gas pressure comprises air pressure. In some embodiments, applying comprises applying the gas pressure around the polymer fiber solution using an air brush. In some embodiments, the polymer fiber solution moves into the air brush via an electromagnetic pressure, an electromechanical pressure, gravity, a hydraulic pressure, a mechanical extrusion pressure, a pneumatic pressure, or a combination thereof. In some embodiments, the polymer fiber solution moves into the air brush via the mechanical extrusion pressure using a syringe pump. In some embodiments, the air brush comprises an inner feed line. In some embodiments, the polymer fiber solution moves inside the inner feed line. In some embodiments, the polymer fiber solution moves inside the inner feed line continuously during the forming. In some embodiments, the air brush comprises an outer air line. In some embodiments, the air moves inside the outer air line. In some embodiments, the air brush comprises a nozzle to eject the polymer fiber solution. In some embodiments, the air brush comprises at least two nozzles to eject the polymer fiber solution. In some embodiments, the nozzle is uniaxial, coaxial, triaxial, tetraaxial, or pentaaxial. In some embodiments, the nozzle is at least about 30 μm. In some embodiments, the nozzle is at least about 300 μm. In some embodiments, the forming comprises at most about 10 hours. In some embodiments, the forming comprises at most about 5 hours. In some embodiments, the polymer fiber comprises a carbohydrate glass, a carbon nanotubes, a chitosan, a collagen, a dextran, a fibrinogen, a fibroin, a gelatin, a glycosaminoglycan, a hydroxyapatite, a keratin, a latex, a nanobioactive glass, a poly(ester-urethane) urea, a poly(ethylene glycol), a poly(ethylene oxide), a poly(lactic acid-co-glycolic acid), a poly(lactide-co-caprolactone), a poly(propylene carbonate), a polyaniline, a polydiaxanone, a polyglycolic acid, a polylactic acid, or a combination thereof. In some embodiments, the collagen comprises a type I collagen or a type II collagen or a type III collagen, or other types of collagens. In some embodiments, the collagen comprises a type I collagen. In some embodiments, the collagen is a methacrylated collagen. In some embodiments, the polymer fiber comprises the glycosaminoglycan. In some embodiments, the method further comprises seeding the scaffold with a cell. In some embodiments, the cell comprises a chondrocyte, an endothelial cell, a pericyte, a meniscus cell, an osteoblast, an osteocyte, an osteoclast, a mesenchymal cell, or a synovial cell. In some embodiments, the cell comprises a stem cell including embryonic stem cells and mesenchymal stem cells. In some embodiments, the stem cell comprises an Infrapatellar fat pad (IPFP)-derived stem cell. In some embodiments, the seeding comprises a plurality of cells. In some embodiments, the plurality of cells comprises at least about 1×10\hat{ }3 cells. In some embodiments, the plurality of cells comprises at least about 5×10\hat{ }6 cells. In some embodiments, the scaffold is suitable for implantation. In some embodiments, the scaffold comprises a Young's modulus. In some embodiments, the Young's modulus is about 10 MPa to about 1000 MPa. In some embodiments, the Young's modulus is about 1 MPa to about 2000 MPa. In some embodiments, the method further comprises culturing the cell seeded in the scaffold. In some embodiments, the method further comprises producing a plurality of polymer scaffold. In some embodiments, the method further comprises combining the plurality of polymer scaffold compositions to form a multilayer construct

In another aspect, there are provided methods comprising applying a gas pressure around a polymer fiber solution comprising a plurality of polymer fibers to eject the polymer fiber solution; rotating a collector; collecting at least a subset of the plurality of polymer fibers onto a surface of the collector; crosslinking the at least the subset of the plurality of polymer fibers, wherein the scaffold is suitable for use as a meniscal repair device, wherein the collector is rotated on an axis co-linear to an ejection path the applying. In some embodiments, the crosslinking comprises chemical crosslinking, photo-crosslinking, physical crosslinking, or a combination thereof. In some embodiments, the crosslinking of comprises the chemical crosslinking. In some embodiments, the chemical crosslinking comprises a chemical crosslinking agent. In some embodiments, the chemical crosslinking agent comprises an aldehyde, a calcium chloride, a genipin, a glutaraldehyde, a Trout's reagent, or a combination thereof. In some embodiments, the chemical crosslinking agent comprises the glutaraldehyde. In some embodiments, the crosslinking comprises the photo-crosslinking. In some embodiments, the photo-crosslinking comprises a photo-crosslinking agent. In some embodiments, the photo-crosslinking agent comprises Eosin Y, Irgacure 2959, VA-086, lithium phenyl-2,4,6-trimethylbenzoylphosphinate (LAP), or a combination thereof. In some embodiments, the scaffold has a shape of a bone, a cartilage, a fibrous tissue, a muscle, or a combination thereof. In some embodiments, the fibrous tissue comprises a band, a blood vessel, a bursas, a labrum, a ligament, a meniscus, a tendon, or a combination thereof. In some embodiments, the collector comprises a mesh collector or a solid collector. In some embodiments, the collector rotates at least about 10 revolutions per minute (rpm). In some embodiments, the collector rotates at least about 600 rpm. In some embodiments, the collector rotates at a constant speed. In some embodiments, the collector rotates at a varying speed. In some embodiments, the gas pressure is at least about 1 pound per square inch (psi). In some embodiments, the gas pressure is at least about 10 psi. In some embodiments, the gas pressure is at least about 30 psi. In some embodiments, the gas pressure comprises air pressure. In some embodiments, the applying comprises applying the gas pressure around the polymer fiber solution using an air brush. In some embodiments, the polymer fiber solution moves into the air brush via an electromagnetic pressure, an electromechanical pressure, gravity, a hydraulic pressure, a mechanical extrusion pressure, a pneumatic pressure, or a combination thereof. In some embodiments, the polymer fiber solution moves into the air brush via the mechanical extrusion pressure using a syringe pump. In some embodiments, the air brush comprises an inner feed line. In some embodiments, the polymer fiber solution moves inside the inner feed line. In some embodiments, the polymer fiber solution moves inside the inner feed line continuously during the collecting. In some embodiments, the air brush comprises an outer air line. In some embodiments, the air moves inside the outer air line. In some embodiments, the air brush comprises a nozzle to eject the polymer fiber solution. In some embodiments, the air brush comprises at least two nozzles to eject the polymer fiber solution. In some embodiments, the nozzle is uniaxial, coaxial, triaxial, tetraaxial, or pentaaxial. In some embodiments, the nozzle is at least about 30 μm. In some embodiments, the nozzle is at least about 300 μm. In some embodiments, the forming comprises at most about 10 hours. In some embodiments, the forming comprises at most about 5 hours. In some embodiments, the plurality of polymer fibers comprises a carbohydrate glass, a carbon nanotubes, a chitosan, a collagen, a dextran, a fibrinogen, a fibroin, a gelatin, a glycosaminoglycan, a hydroxyapatite, a keratin, a latex, a nanobioactive glass, a poly(ester-urethane) urea, a poly(ethylene glycol), a poly(ethylene oxide), a poly(lactic acid-co-glycolic acid), a poly(lactide-co-caprolactone), a poly(propylene carbonate), a polyaniline, a polydiaxanone, a polyglycolic acid, a polylactic acid, or a combination thereof. In some embodiments, the collagen comprises a fiber a type I collagen or a type II collagen. In some embodiments, the collagen comprises a type I collagen. In some embodiments, the collagen is a methacrylated collagen. In some embodiments, the plurality of polymer fibers comprises the glycosaminoglycan. In some embodiments, the method further comprises seeding the scaffold with a cell. In some embodiments, the cell comprises a chondrocyte, an endothelial cell, a meniscus cell, a mesenchymal cell, or a synovial cell. In some embodiments, the cell comprises a stem cell. In some embodiments, the stem cell comprises an Infrapatellar fat pad (IPFP)-derived stem cell. In some embodiments, the seeding comprises a plurality of cells. In some embodiments, the plurality of cells comprises at least about 1×10\hat{ }3 cells. In some embodiments, the plurality of cells comprises at least about 5×10\hat{ }6 cells. In some embodiments, the method further comprises culturing the cell seeded in the scaffold. In some embodiments, the method further comprises producing a plurality of polymer scaffold. In some embodiments, the method further comprises combining the plurality of polymer scaffold compositions to form a multilayer construct. In some embodiments, the scaffold is suitable for implantation. In some embodiments, the scaffold comprises a Young's modulus. In some embodiments, the Young's modulus is about 10 MPa to about 1000 MPa. In some embodiments, the Young's modulus is about 1 MPa to about 2000 MPa.

In another aspect, there are provided methods comprising applying a gas pressure around a polymer fiber solution comprising a plurality of polymer fibers to eject the polymer fiber solution; rotating a collector; collecting at least a subset of the plurality of polymer fibers onto the collector; crosslinking the at least the subset of the plurality of polymer fibers; forming a scaffold comprising the at least the subset of the plurality of polymer fibers, wherein the collector comprises a shape of a meniscus, wherein the collector is rotated on an axis co-linear to an ejection path, wherein the scaffold is suitable for use as a meniscal repair device and wherein the scaffold has a thickness of at least about 300 micrometer (μm) and a porosity of at least about 40%.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIGS. 1A-1D depict the pneumatospinning setup. FIG. 1A depicts a photograph of an airbrush and mesh collector. FIG. 1B is a diagram of a polymer jet generated by air flow created by using the pneumatospinning setup. FIG. 1C is a photograph of a scaffold that was spun on a mesh collector. FIG. 1D depicts a scaffold that was cut to the shape of a human meniscus.

FIGS. 2A-2C depict mechanical properties of pneumatospun collagen scaffolds that were deposited upon a spinning mesh collector. FIG. 2A depicts the manner in which scaffold specimens were harvested from the mesh collector in either in the circumferential or radial directions in relation to the rotation of the scaffold. FIG. 2B is a graph showing the elastic modulus of dry collagen scaffolds after glutaraldehyde crosslinking. FIG. 2C is a graph showing the elastic modulus of dry collagen scaffolds after glutaraldehyde crosslinking following hydration in medium.

FIGS. 3A-3D depict scanning electron micrographs (SEM) of collagen scaffolds after glutaraldehyde crosslinking. FIG. 3A and FIG. 3B depict pneumatospun scaffolds. FIG. 3C and FIG. 3D depict electrospun scaffolds.

FIGS. 4A-4H illustrate results of cell viability, histology, and gene expression of IPFP-MSC seeded pneumatospun collagen scaffolds. FIGS. 4A-4B show cell viability of IPFP-MSC seeded collagen scaffolds (live cells=green, dead cells=red, bar=1000 μm) using the Live/Dead kit (Invitrogen, Waltham, MA). FIG. 4C shows Safranin O stain of neotissue formed on a collagen scaffold. FIG. 4D displays Collagen type I immunostaining (AP red) on a collagen scaffold. FIG. 4E shows Collagen type II immunostaining (DAB brown) on a collagen scaffold. FIG. 4F shows Isotype control immunostaining. (Bar=200 μm). FIG. 4G and FIG. 4H show graphs of gene expression levels of IPFP-MSC seeded on collagen scaffolds after 3 weeks in serum free medium relative to monolayer undifferentiated cultured IPFP-MSC (N=6) for the genes COL1A1, COL2A1, COMP, THY-1 and ACAN.

FIGS. 5A-5H depict pneumatospun collagen scaffolds that were seeded with human IPFP cells and implanted into ex vivo bovine meniscus and cultured for 3 weeks. FIG. 5A and FIG. 5B are photographs of implanted collagen scaffolds. FIG. 5C shows Safranin O staining in an implanted collagen scaffold. FIG. 5D shows Collagen type I immunostaining in an implanted collagen scaffold. FIG. 5E shows Collagen type II immunostaining in an implanted collagen scaffold. FIG. 5F shows the results from an isotype control. FIG. 5G and FIG. 5H illustrate a higher magnification of a Safranin O stained image showing integration of neotissue (nt) with host tissue (ht). The collagen scaffold is stained dark blue (FIG. 5C-5F Bar=200 m; FIG. 5G-5H Bar=50 μm).

FIGS. 6A-6C depict neumatospun collagen scaffolds seeded with human IPFP cells and implanted into human OA meniscus explant (72-year-old female) and cultured for 3 weeks. FIG. 6A is a photograph of implanted scaffold. FIG. 6B shows an overview of section that has been stained with Safranin O showing neotissue development in meniscus defect (bar=500 μm). FIG. 6C is a composite of histologic images of sections stained with Safranin O and immunostained with collagen type I (AP red), collagen type II (DAB brown), and isotype control regions of inset regions outlined in FIG. 6B (bar=300 μm).

FIGS. 7A-7D display photographs of different mesh collector shapes. FIG. 7A shows a flat mesh collector. FIG. 7B shows a curved mesh collector. FIG. 7C and FIG. 7D display different curved 3D shaped mesh collectors.

FIGS. 8A and 8B depict cell viability of IPFP-MSC seeded pneumatospun collagen scaffolds after overnight incubation on an orbital shaker. FIG. 8A shows live cells (in green) using the Live/Dead kit (Invitrogen, Waltham, MA). FIG. 8B shows dead cells (in red) using the Live/Dead kit (Invitrogen, Waltham, MA).

FIGS. 9A-9C depict composite mats of electrospun PLA and pneumatospun collagen. The composite approach leverages the mechanical strength of the PLA and the biocompatibility and porosity of the collagen. FIG. 9A shows a photograph of a composite scaffold of PLA and collagen. FIG. 9B shows a scanning electron micrograph (SEM) of aligned PLA mat fibers (bottom layer). FIG. 9C shows a SEM of aligned pneumatospun collagen mat (top layer).

FIGS. 10A-10D depict the histology for pneumatospun collagen scaffolds seeded with ES-MSC. FIG. 10A shows the control collagen scaffolds with low to negative Safranin O (GAG positive) (bar=200 μm). FIG. 10B shows the immunopositive signal for collagen type I, indicating a vascular meniscus tissue phenotype (bar=200 μm). FIG. 10C shows a more extensive GAG staining (bar=200 μm). FIG. 10D shows a lower signal for collagen type I, which is consistent with an avascular-like meniscus tissue (bar=200 μm).

FIG. 11 depicts graphs showing the gene expression profiles of ES-MSC cultured upon blow spun collagen scaffolds without (control) and with herparin conjugation and conjugated growth factors. The genes shown on the graphs include COL1A1, COL2A1, CHAD, THY-1, COMP, and MKX.

FIGS. 12A-12D depict images of histology staining in heparinized and TGFβ1 conjugated pneumatospun collagen scaffolds seeded with ES-MSC and implanted into OA human tissue defects and cultured in scrum free medium with TGFβ3 for 6 weeks. FIG. 12A shows the histology staining of glycosaminoglycan deposition (Safranin O positive) (bar=400 μm). FIG. 12B and FIG. 12C show degree staining indicating production of extracellular matrix with meniscus-like collagen type I and II. FIG. 12B shows the histology staining of collagen type I (bar=400 μm). FIG. 12C shows the histology staining of collagen type II (bar=400 μm). FIG. 12D shows the histology of the isotype control showing little background immunostaining (bar=400 μm).

### DETAILED DESCRIPTION

Menisci are an essential tissue in knee joints that contribute to load distribution, knee stability, and protect articular cartilage during normal activity. Injuries to menisci are common, with an annual estimated incidence between 600,000 and 850,000 in the United States alone, 90% of which require surgical intervention. In some embodiments, tears occurring in the vascular zone have a higher rate of repair via surgery. In some embodiments, very few tears in the avascular zone heal, and around a third of repairs fail. Smaller tears are frequently repaired using sutures, screws, arrows, or darts. However, in some cases, many tears are too complex or extensive to repair, and thus, most surgeries involve partial, subtotal, or total meniscectomy. In some embodiments, loss of meniscal tissue alters joint loading dynamics, leading to joint destabilization and finally a progression to osteoarthritis (OA). To address large tears, or for the replacement of menisci, meniscal substitutions have been proposed for partial and total meniscus replacement.

Efforts towards engineering meniscus tissues typically combine cells with a variety of diverse natural and synthetic scaffolds. However, a central feature that underlies the load bearing role of meniscus is the unique organization of collagen fibers, which is an essential requirement for a functional engineered meniscus. The technique of electrospinning(ES) has been applied to create nanofibrous scaffolds that emulate the meniscus collagen fibrillar matrix using natural and synthetic polymer.

Examples of ES scaffolds explored for meniscus regeneration include synthetic polymers such as PLA, PCL, natural polymers such as collagen, or combinations such as PCL and silk fibroin, PLGA and gelatin, or PLA and collagen. These electrospun scaffolds were biocompatible, supported attachment of a variety of cell types including meniscus fibroblasts, various sources of MSC and synovial cells, and lead to neo-tissue formation and repair of ex vivo meniscal tears. Electrospun scaffolds have shown promise in terms of cytocompatibility and neotissue formation. However, electrospinning often requires days to generate a scaffold of even a few millimeters in thickness. Layering of ES scaffolds or incorporating ES collagen micro/nanofibers within a macroporous PLA/PLGA foam have been described to generate thicker constructs. However, the issue of delamination under loading remains a concern. Moreover, the highly dense nature of the fibers comprising the scaffold hinder efficient cell seeding and migration throughout the scaffold. To overcome this issue, strategies have been adopted to increase scaffold porosity by including water soluble sacrificial fibers, or by incorporating growth factors or chemoattractants within the scaffold to facilitate cell migration, support proliferation and tissue formation.

In aspects of methods herein, solution blow spinning or pneumatospinning is a method of generating fibrous scaffolds. In embodiments, pressurized gas driven through an outer nozzle generates a stream of polymer solution fed through the inner nozzle of a coaxial system. Pneumatospinning relies on gas pressure instead of an electric charge, which, in some cases, facilitates the fabrication of thicker constructs without the insulating effect that limits the thickness of electrospun constructs. In some embodiments, pneumatospinning is simpler, safer, more efficient, and can be achieved with less expensive and commercially available tools and therefore has the potential to overcome some of the limitations of electrospinning for meniscal repair.

In various aspects of methods herein, a wide range of materials are contemplated to be pneumatospun to create nano- and microfibrous scaffolds. Pneumatospinning of synthetic polymers such as polyurethane, polymethylmethacrylate, polyvinyl alcohol, polylactic acid, and polycaprolactone is used in some cases. In some embodiments, natural polymers such as silk fibroin and collagen are pneumatospun.

Provided herein are methods of making pneumatospun bioscaffolds. In embodiments, bioscaffolds suitable for making using methods herein include, but are not limited to, meniscus, tendon, bursa, bands, ligament, labrum, bone, cartilage, muscle, skin, kidney, liver, heart, lung, trachea, bronchi, blood vessels, intestine, brain, spinal cord, nerves, pancreas, and tumor organoids. Pneumatospinning offers a safe, convenient, and efficient method of generating tissues with a desired shape and structural properties, such as bone, cartilage, muscle, meniscus, tendons, ligaments, labrum, and blood vessels. In addition, pneumatospinning can be used effectively to encapsulate other tissues, organs, and organoids for potential clinical implantation, research and development, and drug discovery.

Headers and sub-headers as used herein in this application are for organizational purposes only. The headers and sub-headers are not intended to limit the disclosed inventive concepts in any way.

### Formation of Polymer Fibers

### Pneumatospinning

Disclosed herein, in some embodiments, are methods of pneumatospinning. In some embodiments, pneumatospinning comprises blow spinning. In some embodiments, pneumatospinning comprises solution blow spinning. In some embodiments, pneumatospinning comprises feeding a polymer fiber solution comprising a polymer fiber into an air brush. In some embodiments, pneumatospinning comprises applying a gas pressure around the polymer fiber solution comprising the polymer fiber in the air brush. In some embodiments, pneumatospinning comprises ejecting the polymer fiber from the air brush. In some embodiments, the air brush comprises a nozzle. In some embodiments, the polymer fiber is ejected from the nozzle. In some embodiments, more than one nozzle is used. In some embodiments, the ejecting comprises using air pressure from the air brush. In some embodiments, the ejecting comprises using gravity. In some embodiments, pneumatospinning comprises collecting the polymer fiber by a collector. In some embodiments, pneumatospinning comprises collecting the polymer fiber by a meshed collector. In some embodiments, the meshed collector comprises a meshed material. In some embodiments, the meshed material reduces turbulence.

### Applying a Gas Pressure

Disclosed herein, in some embodiments, are methods comprising applying a gas pressure. In some embodiments, the gas pressure is at least about 1 pound per square inch (psi), about 2 psi, about 3 psi, about 4 psi, about 5 psi, about 6 psi, about 7 psi, about 8 psi, about 9 psi, about 10 psi, about 12 psi, about 14 psi, about 16 psi, about 18 psi, about 20 psi, about 22 psi, about 24 psi, about 26 psi, or at least about 28 psi. In some embodiments, the gas pressure is at least about 30 psi, about 32 psi, about 34 psi, about 36 psi, about 38 psi, about 40 psi, about 42 psi, about 44 psi, about 46 psi, about 48 psi, or at least about 50 psi. In some embodiments, the gas pressure comprises air pressure. In some embodiments, the gas pressure does not comprise air pressure.

### Polymer Fiber

Disclosed herein, in some embodiments, are methods of forming polymer fibers. In some embodiments, the polymer fiber comprises a protein. In some embodiments, the polymer fiber comprises a carbohydrate. In some embodiments, the carbohydrate is a glycosaminoglycan. In some embodiments, the polymer fiber comprises a carbohydrate glass. In some embodiments, the polymer fiber comprises carbon nanotubes. In some embodiments, the polymer fiber comprises a chitosan. In some embodiments, the polymer fiber comprises a collagen. In some embodiments, the polymer fiber comprises a dextran. In some embodiments, the polymer fiber comprises a fibrinogen. In some embodiments, the polymer fiber comprises a fibroin. In some embodiments, the polymer fiber comprises a gelatin. In some embodiments, the polymer fiber comprises a glycosaminoglycan. In some embodiments, the polymer fiber comprises a hydroxyapatite. In some embodiments, the polymer fiber comprises a keratin. In some embodiments, the polymer fiber comprises a latex. In some embodiments, the polymer fiber comprises a nanobioactive glass. In some embodiments, the polymer fiber comprises a poly(ester-urethane) urea. In some embodiments, the polymer fiber comprises a poly(ethylene glycol). In some embodiments, the polymer fiber comprises a poly(ethylene oxide). In some embodiments, the polymer fiber comprises a poly(lactic acid-co-glycolic acid). In some embodiments, the polymer fiber comprises a poly(lactide-co-caprolactone). In some embodiments, the polymer fiber comprises a poly(propylene carbonate). In some embodiments, the polymer fiber comprises a polyaniline. In some embodiments, the polymer fiber comprises a polydiaxanone. In some embodiments, the polymer fiber comprises a polyglycolic acid. In some embodiments, the polymer fiber comprises a polylactic acid. In some embodiments, the polymer fiber comprises a carbohydrate glass, a carbon nanotubes, a chitosan, a collagen, a dextran, a fibrinogen, a fibroin, a gelatin, a glycosaminoglycan, a hydroxyapatite, a keratin, a latex, a nanobioactive glass, a poly(ester-urethane)urea, a poly(ethylene glycol), a poly(ethylene oxide), a poly(lactic acid-co-glycolic acid), a poly(lactide-co-caprolactone), a poly(propylene carbonate), a polyaniline, a polydiaxanone, a polyglycolic acid, a polylactic acid, or a combination thereof.

In some embodiments, the polymer fiber comprises collagen. In some embodiments, the collagen comprises a type I collagen. In some embodiments, the collagen comprises a type II collagen. In some embodiments, the collagen comprises a type I or a type II collagen. In some embodiments, the collagen is selected from type I collagen, type II collagen, type III collagen, type IV collagen, type V collagen, type VI collagen, type VII collagen, type VIII collagen, type IX collagen, type X collagen, type XI collagen, type XII collagen, type XIII collagen, type XIV collagen, type XV collagen, type XVI collagen, type XVII collagen, type XVIII collagen, type XIX collagen, type XX collagen, type XXI collagen, type XXII collagen, type XXIII collagen, type XXIV collagen, type XXV collagen, type XXVI collagen, type XXVII collagen, type XXVIII collagen, or combinations thereof. In some embodiments, the collagen comprises methacrylated collagen. In some embodiments, the collagen does not comprise methacrylated collagen.

In some embodiments, the polymer is dissolved to about a 1 wt % solution, about a 2 wt % solution, about a 4 wt % solution, about a 6 wt % solution, about a 8 wt % solution, about a 10 wt % solution, about a 12 wt % solution, about a 14 wt % solution, about a 15 wt % solution, about a 16 wt % solution, about 17 wt % solution, about a 18 wt % solution, about a 19 wt % solution, about a 20 wt % solution, or about a 22 wt % solution. In some embodiments, the polymer solution comprises type I collagen dissolved to a 16 wt % solution.

In some embodiments, the polymer solution comprises polylactic acid dissolved to about a 1 wt % solution, about a 2 wt % solution, about a 4 wt % solution, about a 6 wt % solution, about a 8 wt % solution, about a 10 wt % solution, about a 12 wt % solution, about a 14 wt % solution, about a 15 wt % solution, about a 16 wt % solution, about 17 wt % solution, about a 18 wt % solution, about a 19 wt % solution, about a 20 wt % solution, or about a 22 wt % solution. In some embodiments, the polymer solution comprises polylactic acid dissolved to a 10 wt % solution.

In some embodiments, the polymer fibers possess an average ultimate tensile strength of between about 0.1 Mpa to about 2000 Mpa after crosslinking. In some embodiments, the polymer fibers possess an average ultimate tensile strength of between about 0.1 Mpa to about 1000 Mpa after crosslinking. In some embodiments, the polymer fibers possess an average ultimate tensile strength of between about 1 Mpa to about 500 Mpa after crosslinking. In some embodiments, the polymer fibers possess an average ultimate tensile strength of between about 1 Mpa to about 100 Mpa after crosslinking. In some embodiments, the polymer fibers possess an average ultimate tensile strength of between about 1 Mpa to about 10 Mpa after crosslinking In some embodiments, the polymer fibers possess an average ultimate tensile strength of between about 10 Mpa to about 20 Mpa, between about 20 Mpa to about 30 Mpa, between about 30 Mpa to about 40 Mpa, between about 40 Mpa to about 50 Mpa, between about 50 Mpa to about 60 Mpa, between about 60 Mpa to about 70 Mpa, between about 70 Mpa to about 80 Mpa, between about 80 Mpa to about 90 Mpa, between about 90 Mpa to about 100 Mpa, between about 100 Mpa to about 110 Mpa, between about 110 Mpa to about 120 Mpa, between about 120 Mpa to about 130 Mpa, between about 130 Mpa to about 140 Mpa, between about 140 Mpa to about 150 Mpa, between about 150 Mpa to about 160 Mpa, between about 160 Mpa to about 170 Mpa, between about 170 Mpa to about 180 Mpa, between about 180 Mpa to about 190 Mpa, between about 190 Mpa to about 200 Mpa, about 250 Mpa, about 300 Mpa, about 350 Mpa, about 400 Mpa or about 500 Mpa after crosslinking.

### Plurality of Polymer Fibers

Disclosed herein, in some embodiments, are methods of forming a plurality of polymer fibers. In some embodiments, the plurality of polymer fibers comprises a protein. In some embodiments, the plurality of polymer fibers comprises a carbohydrate. In some embodiments, the carbohydrate is a glycosaminoglycan. In some embodiments, the plurality of polymer fibers comprises a carbohydrate glass. In some embodiments, the plurality of polymer fibers comprises carbon nanotubes. In some embodiments, the plurality of polymer fibers comprises a chitosan. In some embodiments, the plurality of polymer fibers comprises a collagen. In some embodiments, the plurality of polymer fibers comprises a dextran. In some embodiments, the plurality of polymer fibers comprises a fibrinogen. In some embodiments, the plurality of polymer fibers comprises a fibroin. In some embodiments, the plurality of polymer fibers comprises a gelatin. In some embodiments, the plurality of polymer fibers comprises a glycosaminoglycan. In some embodiments, the plurality of polymer fibers comprises a hydroxyapatite. In some embodiments, the plurality of polymer fibers comprises a keratin. In some embodiments, the plurality of polymer fibers comprises a latex. In some embodiments, the plurality of polymer fibers comprises a nanobioactive glass. In some embodiments, the plurality of polymer fibers comprises a poly(ester-urethane)urea. In some embodiments, the plurality of polymer fibers comprises a poly(ethylene glycol). In some embodiments, the plurality of polymer fibers comprises a poly(ethylene oxide). In some embodiments, the plurality of polymer fibers comprises a poly(lactic acid-co-glycolic acid). In some embodiments, the plurality of polymer fibers comprises a poly(lactide-co-caprolactone). In some embodiments, the plurality of polymer fibers comprises a poly(propylene carbonate). In some embodiments, the plurality of polymer fibers comprises a polyaniline. In some embodiments, the plurality of polymer fibers comprises a polydiaxanone. In some embodiments, the plurality of polymer fibers comprises a polyglycolic acid. In some embodiments, the plurality of polymer fibers comprises a polylactic acid. In some embodiments, the plurality of polymer fibers comprises a carbohydrate glass, a carbon nanotubes, a chitosan, a collagen, a dextran, a fibrinogen, a fibroin, a gelatin, a glycosaminoglycan, a hydroxyapatite, a keratin, a latex, a nanobioactive glass, a poly(ester-urethane)urea, a poly(ethylene glycol), a poly(ethylene oxide), a poly(lactic acid-co-glycolic acid), a poly(lactide-co-caprolactone), a poly(propylene carbonate), a polyaniline, a polydiaxanone, a polyglycolic acid, a polylactic acid, or a combination thereof.

In some embodiments, the plurality of polymer fibers comprises collagen. In some embodiments, the collagen comprises a type I collagen. In some embodiments, the collagen comprises a type II collagen. In some embodiments, the collagen comprises a type I or a type II collagen. In some embodiments, the collagen is selected from type I collagen, type II collagen, type III collagen, type IV collagen, type V collagen, type VI collagen, type VII collagen, type VIII collagen, type IX collagen, type X collagen, type XI collagen, type XII collagen, type XIII collagen, type XIV collagen, type XV collagen, type XVI collagen, type XVII collagen, type XVIII collagen, type XIX collagen, type XX collagen, type XXI collagen, type XXII collagen, type XXIII collagen, type XXIV collagen, type XXV collagen, type XXVI collagen, type XXVII collagen, type XXVIII collagen, or combinations thereof. In some embodiments, the collagen comprises methacrylated collagen. In some embodiments, the collagen does not comprise methacrylated collagen.

### Collector

Disclosed herein, in some embodiments, are methods comprising collecting a polymer fiber by a collector. In some embodiments, are methods comprising collecting at least a subset of a plurality of polymer fibers.

In some embodiments, the collector comprises a curvature on a surface of the collector. In some embodiments, the collector comprises a first curvature and a second curvature on a surface of the collector. In some embodiments, the collector comprises a first curvature and a second curvature on a surface of the collector. In some embodiments, the curvature comprises a degree of curvature. In some embodiments, the curvature comprises a degree of curvature of the first curvature and a degree of curvature of the second curvature. In some embodiments, the degree of curvature of the first curvature and the degree of curvature of the degreed curvature are the same. In some embodiments, the degree of curvature of the first curvature and the degree of curvature of the second curvature are different.

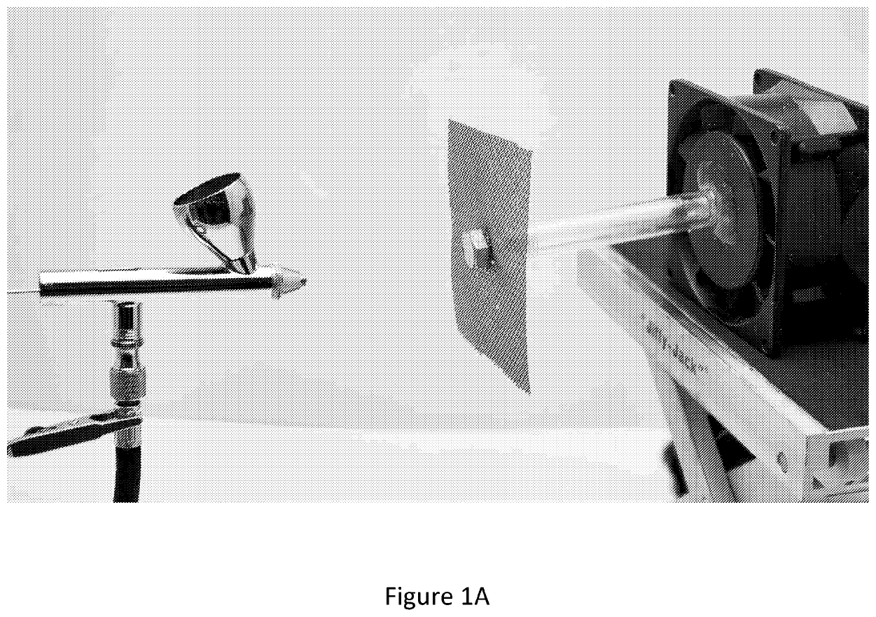

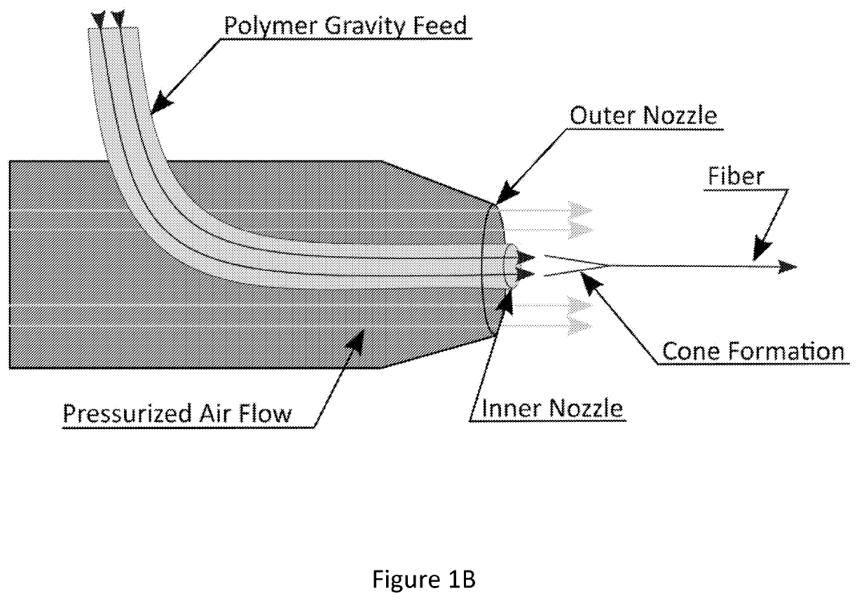

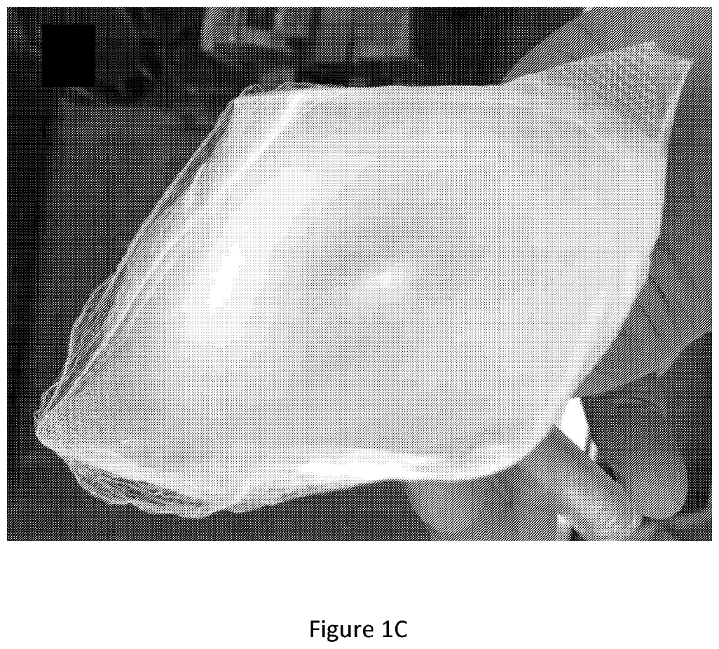

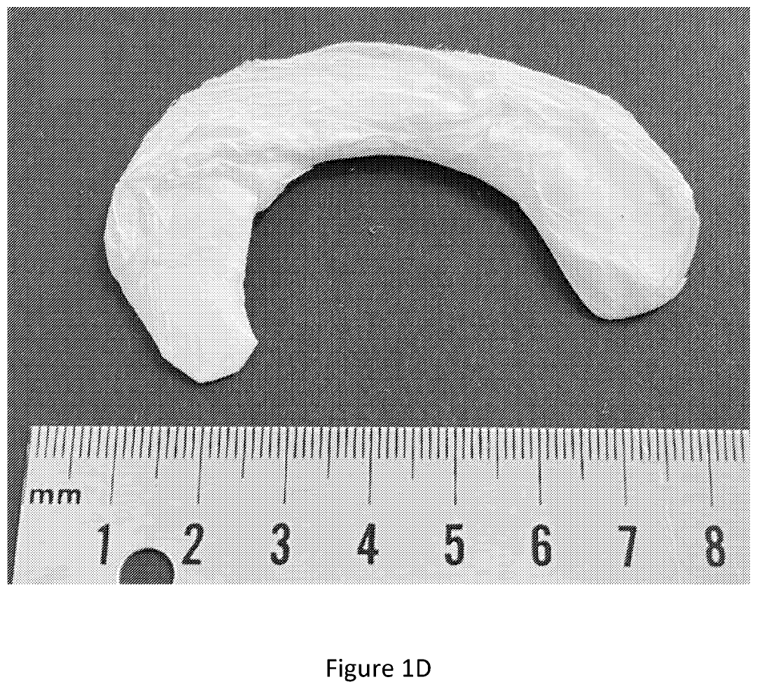

In some embodiments, the collector comprises a mesh collector. For example, FIG. 1C is a photograph of a scaffold that was spun on a mesh collector. In some embodiments, the mesh collector reduces turbulence in the pneumatospinning. In some embodiments, the mesh collector has a high porosity. In some embodiments, the mesh collector has a low porosity. In some embodiments, the collector comprises a solid collector. In some embodiments, the collector comprises a mesh collector or a solid collector, or a combination thereof.

Disclosed herein, in some embodiments, are methods of rotating a collector. In some embodiments, the collector rotates. In some embodiments, the collector rotates at least about 10 revolutions per minute (rpm), about 20 rpm, about 30, rpm, about 40 rpm, about 50 rpm, about 60 rpm, about 70 rpm, about 80 rpm, about 90 rpm, about 100 rpm, about 200 rpm, about 300 rpm, about 400 rpm, or about 500 rpm. In some embodiments, the collector rotates at least about 600 rpm, about 700 rpm, about 800 rpm, about 900 rpm, about 1000 rpm, about 1100 rpm, about 1200 rpm, about 1300 rpm, about 1400 rpm, about 1500 rpm, about 1600 rpm, about 1700 rpm, about 1800 rpm, about 1900 rpm, about 2000 rpm, about 2100 rpm, about 2200 rpm, about 2300 rpm, about 2400 rpm, or about 2500 rpm.

In some embodiments, the collector rotates at a constant speed. In some embodiments, the collector rotates at an alternating speed. In some embodiments, the collector rotates at a variable speed. In some embodiments, the collector rotates, stops rotating, and resumes rotating. In some embodiments, the collector is not rotating and begins rotating. In some embodiments, the collector rotates at a varying speed. In some embodiments, the collector comprises a rotating collector. In some embodiments, the collector comprises a stationary collector. In some embodiments, the collector comprises a translating collector. In some embodiments, the collector comprises a rotating collector, a stationary collector, or a translating collector. In some embodiments, the collector rotates on an axis co-linear to an ejection path of applying a gas pressure around a polymer fiber solution comprising a plurality of polymer fibers to eject the polymer fiber solution.

In some embodiments, the collecting of the polymer fiber by a collector to form a scaffold comprises a collection time of at most about 10 hours, In some embodiments, the collecting comprises a time of at most about 11 hours, about 12 hours, about 13 hours, about 14 hours, about 15 hours, about 16 hours, about 17 hours, about 18 hours, about 19 hours, or a time of at most about 20 hours. In some embodiments, the collecting of the polymer fiber by a collector to form a scaffold comprises a time of at most about 5 hours. In some embodiments, the collecting comprises a time of at most about 5 minutes, about 10 minutes, about 20 minutes, about 30 minutes, about 45 minutes, about 1 hour, about 2 hours, about 3 hours, or at most a time of about 4 hours. In some embodiments, the collecting time comprises at most about 6 hours, about 7 hours, about 8 hours, or at most a time of about 9 hours. In some embodiments, the collecting time is more than 20 hours. In some embodiments, the collecting time is less than 5 minutes.

### Airbrush

In some embodiments, applying a gas pressure around a polymer fiber solution comprising a polymer fiber to eject the polymer fiber solution comprises applying the gas pressure around the polymer fiber solution using an air brush. In some embodiments, applying a gas pressure around a polymer fiber solution comprising a plurality of polymer fibers to eject the polymer fiber solution comprises applying the gas pressure around the polymer fiber solution using an airbrush.

In some embodiments, the polymer fiber solution moves into the air brush via an electromagnetic pressure. In some embodiments, the polymer fiber solution moves into the air brush via an electromechanical pressure. In some embodiments, the polymer fiber solution moves into the air brush via gravity. In some embodiments, the polymer fiber solution moves into the air brush via a hydraulic pressure. In some embodiments, the polymer fiber solution moves into the air brush via a mechanical extrusion pressure. In some embodiments, the polymer fiber solution moves into the air brush via a pneumatic pressure. In some embodiments, the polymer fiber solution moves into the air brush via an electromagnetic pressure, an electromechanical pressure, gravity, a hydraulic pressure, a mechanical extrusion pressure, a pneumatic pressure, or a combination thereof.

In some embodiments, a syringe pump is used to deliver the polymer fiber solution into the airbrush. In some embodiments, the polymer fiber solution moves into the air brush via a mechanical extrusion pressure. In some embodiments, the polymer fiber solution moves into the airbrush via the mechanical extrusion pressure using a syringe pump. In some embodiments, the polymer fiber solution moves into the airbrush via the mechanical extrusion pressure not using a syringe pump. In some embodiments, other delivery methods are used to deliver the polymer fiber solution into the airbrush. In some embodiments, the other deliver methods used to deliver the polymer fiber solution into the airbrush include use of electromagnetic pressure, an electromechanical pressure, gravity, a hydraulic pressure, a mechanical extrusion pressure, a pneumatic pressure, or a combination thereof.

In some embodiments, the air brush comprises an inner feed line. In some embodiments, the polymer fiber solution moves inside the inner feed line.

In some embodiments, the polymer fiber solution moves inside the inner feed line continuously during the collecting of the polymer fiber by a collector to form a scaffold. In some embodiments, the polymer fiber solution moves inside the inner feed line continuously during the collecting of at least a subset of the plurality of polymer fibers onto a surface of the collector. In some embodiments, the polymer fiber solution moves inside the inner feed line intermittently during the collection of the polymer fiber by a collector to form a scaffold. In some embodiments, the polymer fiber solution moves inside the inner feed line intermittently during the collecting of at least a subset of the plurality of polymer fibers onto a surface of the collector. In some embodiments, the polymer fiber solution moves inside the inner feed line continuously during the formation of a scaffold comprising the polymer. In some embodiments, the polymer fiber solution moves inside the inner feed line intermittently during the formation of a scaffold comprising the polymer. In some embodiments, the polymer fiber solution moves inside the inner feed line variably during the collection of the polymer fiber by a collector to form a scaffold.

In some embodiments, the air brush comprises an outer air line. In some embodiments, the air moves inside the outer air line. In some embodiments, the air moves outside the outer air line.

### Nozzle

In some embodiments, the air brush comprises a nozzle. In some embodiments, the air brush comprises a nozzle to eject the polymer fiber solution. In some embodiments, the air brush comprises at least two nozzles. In some embodiments, the air brush comprises at least two nozzles to eject the polymer fiber solution. In some embodiments, the air brush comprises at least 3, at least 4, at least 5, at least 6, at least 7, at least 8, at least 9, or at least 10 nozzles. In some embodiments, the nozzle is uniaxial. In some embodiments, the nozzle is coaxial. In some embodiments, the nozzle is triaxial. In some embodiments, the nozzle is tetraaxial. In some embodiments, the nozzle is pentaaxial. In some embodiments, the nozzle is uniaxial, coaxial, triaxial, tetraaxial, or pentaaxial, or combinations thereof. In some embodiments, the nozzles are uniaxial, coaxial, triaxial, tetraaxial, or pentaaxial, or combinations thereof.

In some embodiments, the nozzle inner diameter is at least about 30 μm, about 40 μm, about 50 μm, about 60 μm, about 70 μm, about 80 μm, about 90 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or at least about 300 μm. In some embodiments, the nozzle inner diameter is at least about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or at least about 500 μm. In some embodiments, the nozzle inner diameter is less than about 30 μm, about 40 μm, about 50 μm, about 60 μm, about 70 μm, about 80 μm, about 90 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or at least about 300 μm. In some embodiments, the nozzle inner diameter is at least about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or less than about 500 μm.

### Scaffold

Disclosed herein, in some embodiments, are methods of collecting the polymer fiber by a collector to form a scaffold. In some embodiments, are methods of forming a scaffold comprising at least a subset of a plurality of polymer fibers. In some embodiments, the scaffold comprises the shape of the meniscus. In some embodiments, the scaffold comprises a shape of a human meniscus. For example, FIG. 1D depicts a scaffold that was cut to the shape of a human meniscus.

In some embodiments, the scaffold is suitable for use as a meniscal repair device. In some embodiments, the scaffold is suitable for implantation. In some embodiments, the scaffold is suitable for implantation to a joint. In some embodiments, the joint is located in a knee. In some embodiments, the joint is located in an elbow, a wrist, a neck, a jaw, a clavicle, a shoulder blade, a hip, a shoulder, a knee, an ankle, a foot, a spinal column, a finger or a toe.

In some embodiments, the scaffold comprises a Young's modulus. In some embodiments, the Young's modulus is about 10 Mpa to about 1000 Mpa. In some embodiments, the Young's modulus is about 1 Mpa to about 2000 Mpa. In some embodiments, the scaffold comprises a Young's modulus of between about 10 Mpa to about 20 Mpa, between about 20 Mpa to about 30 Mpa, between about 30 Mpa to about 40 Mpa, between about 40 Mpa to about 50 Mpa, between about 50 Mpa to about 60 Mpa, between about 60 Mpa to about 70 Mpa, between about 70 Mpa to about 80 Mpa, between about 80 Mpa to about 90 Mpa, between about 90 Mpa to about 100 Mpa, between about 100 Mpa to about 110 Mpa, between about 110 Mpa to about 120 Mpa, between about 120 Mpa to about 130 Mpa, between about 130 Mpa to about 140 Mpa, between about 140 Mpa to about 150 Mpa, between about 150 Mpa to about 160 Mpa, between about 160 Mpa to about 170 Mpa, between about 170 Mpa to about 180 Mpa, between about 180 Mpa to about 190 Mpa, between about 190 Mpa to about 200 Mpa, about 250 Mpa, about 300 Mpa, about 350 Mpa, about 400 Mpa or about 500 Mpa In some embodiments, the Young's modulus is between about 500 Mpa to about 600 Mpa, between about 600 Mpa to about 800 Mpa, between about 800 Mpa to about 1000 Mpa, between about 1000 Mpa to about 1200 Mpa, between about 1200 Mpa to about 1400 Mpa, between about 1400 Mpa to about 1600 Mpa, between about 1600 Mpa to about 1800 Mpa, or between about 1800 Mpa to about 2000 Mpa.

Disclosed herein, are methods of forming a scaffold comprising the polymer, wherein the scaffold has a thickness of at least about 300 micrometer (μm) and a porosity of at least about 40%. In some embodiments, are methods of forming a scaffold comprising at least a subset of a plurality of polymer fibers, wherein the scaffold has a thickness of at least about 300 micrometer (μm) and a porosity of at least about 40%.

### Scaffold Thickness

In some embodiments, the scaffold has a thickness of at least about 60 μm, at least about 80 μm, at least about 100 μm, at least about 120 μm, at least about 140 μm, at least about 160 μm, at least about 180 μm, at least about 200 μm, at least about 220 μm, at least about 240 μm, at least about 260 μm, or at least about 280 μm. In some embodiments, the scaffold has a thickness of at least about 320 μm, at least about 340 μm, at least about 360 μm, at least about 380 μm, at least about 400 μm, at least about 420 μm, at least about 440 μm, at least about 460 μm, at least about 480 μm, at least about 500 μm, at least about 520 μm, at least about 540 μm, at least about 560 μm, at least about 600 μm, at least about 620 μm, or at least about 640 μm. In some embodiments, the scaffold has a thickness more than 640 μm. In some embodiments, the scaffold has a thickness less than 60 μm.

In some embodiments, the scaffold has a thickness of at least about 1 millimeter (mm). In some embodiments, the scaffold has a thickness of at least about 0.1 mm, 0.2 mm, 0.3 mm, 0.4 mm, 0.5 mm, 0.6 mm, 0.7 mm, 0.8 mm, or at least 0.9 mm. In some embodiments, the scaffold has a thickness of at least about 2 mm, 3 mm, 4 mm, 5 mm, 6 mm, 7 mm, 8 mm, 9 mm, or at least 10 mm. In some embodiments, the scaffold has a thickness more than 10 mm. In some embodiments, the scaffold has a thickness less than 1 mm.

### Scaffold Porosity

In some embodiments, the scaffold has a porosity of at least about 50%. In some embodiments, the scaffold has a porosity of at least 5%, at least 10%, at least 15%, at least 20%, at least 25% at least 30%, at least 35%, at least 40%, or at least 45% porosity. In some embodiments, the scaffold has a porosity of at least 55%, at least 60%, at least 65%, at least 70%, at least 75%, at least 80%, at least 85%, at least 90%, or at least 95% porosity. In some embodiments, the scaffold has a porosity of 50%. In some embodiments, the scaffold has a porosity less than 50%. In some embodiments, the scaffold has a porosity more than 50%.

### Scaffold Shape

In some embodiments, the scaffold comprises the shape of the meniscus. In some embodiments, the scaffold comprises a shape of a human meniscus. In some embodiments, the scaffold has a shape of a bone. In some embodiments, the scaffold has a shape of a cartilage. In some embodiments, the scaffold has a shape of a fibrous tissue. In some embodiments, the scaffold has the shape of a muscle. In some embodiments, the scaffold has a shape of a bone, a cartilage, a fibrous tissue, a muscle, or a combination thereof. In some embodiments, the fibrous tissue comprises a band. In some embodiments, the fibrous tissue comprises a blood vessel. In some embodiments, the fibrous tissue comprises a bursas. In some embodiments, the fibrous tissue comprises a labrum. In some embodiments, the fibrous tissue comprises a meniscus. In some embodiments, the fibrous tissue comprises a tendon. In some embodiments, the fibrous tissue comprises a band, a blood vessel, a bursas, a labrum, a ligament, a meniscus, a tendon, or a combination thereof.

### Forming of Scaffold

In some embodiments, the forming of a scaffold comprising the polymer fiber comprises a formation time of at most about 10 hours. In some embodiments, the forming of a scaffold comprising the polymer fiber comprises a formation time of at most about 5 hours. In some embodiments, the forming of a scaffold comprising the polymer fiber comprises a formation time of at most about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours about 15 hours, about 16 hours, about 17 hours, about 18 hours, about 19 hours, or at most about 20 hours. In some embodiments, the forming of a scaffold comprising the polymer fiber comprises a formation time of at least about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, or at least about 1 hour.

In some embodiments, the methods further comprise producing a plurality of polymer scaffold compositions. In some embodiments, the methods further comprise producing the plurality of polymer scaffold compositions simultaneously. In some embodiments, the methods further comprise producing the plurality of polymer scaffold compositions sequentially. In some embodiments, the methods further comprise combining the plurality of polymer scaffold compositions to form a multilayer construct In some embodiments, the methods comprise producing the plurality of polymer scaffold compositions separately and combining the plurality of polymer scaffold compositions on a surface that is not the collector to form the multilayer construct. In some embodiments, combining the plurality of polymer scaffold compositions to form the multilayer construct comprises producing the plurality of polymer scaffold compositions sequentially on one collector In some embodiments, the methods comprise combining 2, 3, 4, 5, 6, 7, 8, 9, 10 or more polymer scaffold compositions to form the multilayer construct. For example, an electrospun polymer scaffold may be combined with a pneumatospun polymer scaffold to form a multilayer construct or more than one electrospun polymer scaffolds may be combined with more than one pneumatospun polymer scaffolds. In some embodiments, the methods further comprise combining at least three polymer scaffold compositions to form the multilayer construct. In some embodiments, the methods comprise combining about 10, about 20, about 30, about 40, about 50, about 60, about 70, about 80, about 90, or about 100 polymer scaffold compositions to form the multilayer construct In some embodiments, the methods further comprise combining about 200, about 300, about 400, about 500, about 600, about 700, about 800, about 900 or about 1000 polymer scaffold compositions to form the multilayer construct. In some embodiments, the methods further comprise combining about 10,000, about 100,000, or about 1,000,000 polymer scaffold compositions to form the multilayer construct

### Methods of Producing a Cell-Seeded Scaffold

Disclosed herein, in some embodiments, are methods of i) applying a gas pressure around a polymer fiber solution comprising a polymer fiber to eject the polymer fiber solution; ii) collecting the polymer fiber by a collector to form a scaffold, wherein the collector comprises a shape of a meniscus and iii) seeding the scaffold with a cell.

### Cells

Disclosed herein, in some embodiments, are methods comprising a cell. In some embodiments, the cell comprises a chondrocyte, an osteoblast, an osteoclast, an osteocyte, an endothelial cell, a meniscus cell, a mesenchymal cell, or a synovial cell, or combinations thereof. In some embodiments, the cell comprises a stem cell including mesenchymal stem cells, embryonic stem cells, and pluripotent stem cells. In some embodiments, the stem cell comprises an Infrapatellar fat pad (IPFP)-derived stem cell.

Disclosed herein, in some embodiments, are methods of seeding the scaffold with a cell, wherein the seedling comprises a plurality of cells. In some embodiments, the plurality of cells comprises at least about 1×10\hat{ }3 cells, about 1×10\hat{ }4 cells, about 1×10\hat{ }5 cells, about 1×10\hat{ }6 cells, about 1×10\hat{ }7 cells, about 1×10\hat{ }8 cells, about 1×10\hat{ }9 cells, about 1×10\hat{ }10 cells, about 2×10\hat{ }3 cells, about 2×10\hat{ }4 cells, about 2×10\hat{ }5 cells, about 2×10\hat{ }6 cells, about 2×10\hat{ }7 cells, about 2×10\hat{ }8 cells, about 2×10\hat{ }9 cells, about 2×10\hat{ }10 cells, about 3×10\hat{ }3 cells, about 3×10\hat{ }6 cells, about 3×10\hat{ }9 cells, about 4×10\hat{ }3 cells, about 4×10\hat{ }6 cells, about 4×10\hat{ }9 cells, or about 5×10\hat{ }3 cells. In some embodiments, the plurality of cells comprises at least about 5×10\hat{ }6 cells, about 5×10\hat{ }9 cells, about 6×10\hat{ }3 cells, about 6×10\hat{ }6 cells, about 6×10\hat{ }9 cells, about 7×10\hat{ }3 cells, or at least about 7×10\hat{ }6 cells. In some embodiments, the plurality of cells comprises less than about 1×10\hat{ }3 cells. In some embodiments, the plurality of cells comprises at least about 1×10\hat{ }3 cells, about 1×10\hat{ }4 cells, about 1×10\hat{ }5 cells, about 1×10\hat{ }6 cells, about 1×10\hat{ }7 cells, about 1×10\hat{ }8 cells, about 1×10\hat{ }9 cells, about 1×10\hat{ }10 cells, about 2×10\hat{ }3 cells, about 2×10\hat{ }4 cells, about 2×10\hat{ }5 cells, about 2×10\hat{ }6 cells, about 2×10\hat{ }7 cells, about 2×10\hat{ }8 cells, about 2×10\hat{ }9 cells, about 2×10\hat{ }10 cells, about 3×10\hat{ }3 cells, about 3×10\hat{ }6 cells, about 3×10\hat{ }9 cells, about 4×10\hat{ }3 cells, about 4×10\hat{ }6 cells, about 4×10\hat{ }9 cells, or about 5×10\hat{ }3 cells on half of the scaffold, less than half of the scaffold, or more than half of the scaffold. In some embodiments, the plurality of cells comprises at least about 5×10\hat{ }6 cells, about 5×10\hat{ }9 cells, about 6×10\hat{ }3 cells, about 6×10\hat{ }6 cells, about 6×10\hat{ }9 cells, about 7×10\hat{ }3 cells, or at least about 7×10\hat{ }6 cells on half of the scaffold, less than half of the scaffold, or more than half of the scaffold. In some embodiments, the plurality of cells comprises at least about 1×10\hat{ }3 cells, about 1×10\hat{ }4 cells, about 1×10\hat{ }5 cells, about 1×10\hat{ }6 cells, about 1×10\hat{ }7 cells, about 1×10\hat{ }8 cells, about 1×10\hat{ }9 cells, about 1×10\hat{ }10 cells, about 2×10\hat{ }3 cells, about 2×10\hat{ }4 cells, about 2×10\hat{ }5 cells, about 2×10\hat{ }6 cells, about 2×10\hat{ }7 cells, about 2×10\hat{ }8 cells, about 2×10\hat{ }9 cells, about 2×10\hat{ }10 cells, about 3×10\hat{ }3 cells, about 3×10\hat{ }6 cells, about 3×10\hat{ }9 cells, about 4×10\hat{ }3 cells, about 4×10\hat{ }6 cells, about 4×10\hat{ }9 cells, or about 5×10\hat{ }3 cells per 1 cm×1 cm of the scaffold, more than per 1 cm×1 cm of the scaffold, or less than per 1 cm×1 cm of the scaffold. In some embodiments, the plurality of cells comprises at least about 5×10\hat{ }6 cells, about 5×10\hat{ }9 cells, about 6×10\hat{ }3 cells, about 6×10\hat{ }6 cells, about 6×10\hat{ }9 cells, about 7×10\hat{ }3 cells, or at least about 7×10\hat{ }6 cells per 1 cm×1 cm of the scaffold, more than per 1 cm×1 cm of the scaffold, or less than per 1 cm×1 cm of the scaffold.

In some embodiments, the plurality of cells are selected from keratinocytes, exocrine secretory epithelial cells, hormone secreting cells, epithelial cells, neural or sensory cells, photoreceptor cells, muscle cells, extracellular matrix cells, blood cells, cardiovascular cells, endothelial cells, vascular smooth muscle cells, kidney cells, pancreatic cells, immune cells, stem cells, germ cells, nurse cells. Interstitial cells, stellate cells liver cells, gastrointestinal cells, lung cells, tracheal cells, vascular cells, skeletal muscle cells, cardiac cells, skin cells, smooth muscle cells, connective tissue cells, corneal cells, genitourinary cells, breast cells, reproductive cells, endothelial cells, epithelial cells, fibroblasts, myofibroblasts, Schwann cells, adipose cells, bone cells, bone marrow cells, cartilage cells, pericytes, mesothelial cells, cells derived from endocrine tissue, stromal cells, progenitor cells, lymph cells, endoderm-derived cells, ectoderm-derived cells, mesoderm-derived cells, pericytes, progenitors thereof and a combination thereof.

In some embodiments, the plurality of cells are selected from cartilaginous cells, chondrocytes, chondroblasts, connective tissue fibroblasts, tendon fibroblasts, bone marrow reticular tissue fibroblasts, nonepithelial fibroblasts, pericytes, osteoprogenitor cells, osteoblasts, osteoclasts, meniscal cells or a combination thereof.

In some embodiments, the plurality of cells comprises cartilaginous cells. In some embodiments, the cartilaginous cells are vascular. In some embodiments, the cartilaginous cells are avascular. In some embodiments, the plurality of cells comprises meniscal cells. In some embodiments, the meniscal cells are vascular. In some embodiments, the meniscal cells are avascular.

In some embodiments, the plurality of cells is derived from or isolated from a tissue. In some embodiments, the tissue comprises a connective tissue In some embodiments, the connective tissue comprises cartilage. In some embodiments, the connective tissue comprises tendon. In some embodiments, the connective tissue comprises ligament. In some embodiments, the connective tissue comprises fascia. In some embodiments, the connective tissue comprises fat or fat pads. In some embodiments, the connective tissue comprises dura mater.

In some embodiments, the plurality of cells are selected from stem cells, bone marrow stem cells, progenitor cells, totipotent cells, pluripotent cells, induced pluripotent stem cells, undifferentiated cells, differentiated cells, differentiating cells, trans-differentiating cells, cells from an adult, cells from a child, germ cells, umbilical cells, circulating cells, resident cells, adherent cells, malignant cells, tumor cells, proliferating cells, quiescent cells, senescent cells, apoptotic cells, cytokine-producing cells, migrating cells and a combination thereof.

### Culturing the Cells

In some embodiments, the methods comprise culturing the cell seeded in the scaffold. In some embodiments, the methods comprise culturing the cell seeded in the scaffold in the cell culture for less than 24 hours, less than two days, less than three days, or less than a week. In some embodiments, the methods the methods comprise culturing the cell seeded in the scaffold in the cell culture for more than a week. In some embodiments, the methods comprise culturing the cell seeded in the scaffold in the cell culture for about 1 week to about 3 weeks. In some embodiments, the methods comprise culturing the cell seeded in the scaffold in the cell culture for about 2 weeks. In some embodiments, the methods comprise culturing the cell seeded in the scaffold in the cell culture for about one month. In some embodiments, the methods comprise culturing the cell seeded in the scaffold in the cell culture for more than one month. In some embodiments, the methods comprise culturing the cell seeded in the scaffold in the cell culture for more than 5 weeks, more than 6 weeks, more than 7 week, more than 8 weeks, more than 9 weeks, or more than 10 weeks.

### Crosslinking

Disclosed herein, in some embodiments, are methods of crosslinking the at least the subset of the plurality of polymer fibers onto a surface of the collector, the crosslinking comprising chemical crosslinking, photo-crosslinking, physical crosslinking, or a combination thereof.

### Chemical Crosslinking

Disclosed herein, in some embodiments, are methods of crosslinking the polymer fibers. In some embodiments, crosslinking the polymer fibers comprises chemical crosslinking.

In some embodiments, the chemical crosslinking comprises a chemical crosslinking agent. In some embodiments, the chemical crosslinking agent is a liquid. In some embodiments, the chemical crosslinking agent is a gas or a vapor. In some embodiments, the chemical crosslinking agent comprises an aldehyde. In some embodiments, the chemical crosslinking agent comprises a calcium chloride. In some embodiments, the calcium chloride agent is about 50 mM, about 75, mM, about 100 mM, about 125 mM, about 150 mM, about 175 mM or about 200 mM. In some embodiments, the calcium chloride agent is about 120 mM. In some embodiments, the chemical crosslinking agent comprises a genipin. In some embodiments, the chemical crosslinking agent comprises a glutaraldehyde. In some embodiments, the glutaraldehyde agent is between about 0.05% and about 5% why solution in PBS. In some embodiments, the glutaraldehyde agent is between about 0.1% and 1% why solution in PBS. In some embodiments, the glutaraldehyde agent is about 0.25% w/solution in PBS. In some embodiments, the chemical crosslinking agent comprises a Trout's reagent. In some embodiments, the chemical crosslinking agent comprises an aldehyde, a calcium chloride, a genipin, a glutaraldehyde, a Trout's reagent, or a combination thereof.

Photo Crosslinking

Disclosed herein, in some embodiments, are methods of crosslinking the polymer fibers. In some embodiments, the crosslinking comprises photo-crosslinking. In some embodiments, photocrosslinking occurs about 1 time, about 2 times, about 3 times, about 4 times, about 5 times, about 10 times, about 15 times, about 20 times, about 25 times, about 50 times, or about 100 times. In some embodiments, photocrosslinking occurs for less than about 1 sec, about 2 sec, about 3 sec, about 5 sec, about 10 sec, about 15 sec, about 20 sec, about 25 sec, about 30 sec, about 35 sec, about 40 sec, about 45 sec, about 50 sec, about 55 sec, about 60 sec, about 65 sec, about 70 sec, about 75 sec, bout 80 sec, about 85 sec, about 90 sec, about 95 sec, about 100 sec, about 105 sec, about 110 sec, about 115 sec, about 120 sec, about 125 sec, about 130 sec, about 135 sec, about 140 sec, about 145 sec about 150 sec, about 160 sec or about 180 sec. In some embodiments, photocrosslinking occurs for less than about 2 min. In some embodiments, the photo-crosslinking comprises a photo-crosslinking agent. In some embodiments, the photo-crosslinking agent comprises Eosin Y. In some embodiments, the photo-crosslinking agent comprises Irgacure 2959. In some embodiments, the photo-crosslinking agent comprises VA-086. In some embodiments, the photo-crosslinking agent comprises lithium phenyl-2,4,6-trimethylbenzoylphosphinate (LAP). In some embodiments, the photo-crosslinking agent comprises Eosin Y, Irgacure 2959, VA-086, lithium phenyl-2,4,6-trimethylbenzoylphosphinate (LAP), or a combination thereof.

### Physical Crosslinking

Disclosed herein, in some embodiments, are methods of crosslinking the polymer fibers. In some embodiments, the crosslinking comprises physical crosslinking.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Efficient Fabrication of Microfibrous Collagen Scaffolds

This example shows the feasibility and the features of methods to produce pneumatospun collagen scaffolds. Pneumatospinning involves a gravity-fed airbrush which generated undesirable turbulence at traditional collector targets used in electrospinning such as flat plates or solid drums. Therefore, meshed collectors were selected that permit air flow through the target, reduce turbulence, and result in more consistent accumulation of spun fibers. To further reduce turbulence at the target, a fan was mounted behind a stationary collector to direct air through the target and then mounted the target to the spinning fan to induce circumferential alignment of fibers. Collagen type I (Semed S, generously supplied by DSM Biomedical, Exton, PA) was dissolved in HFIP at concentrations between 5% to 10% wt/vol to establish the optimal concentration for spinning. The collagen solution was loaded into a customized gravity feed airbrush with a 350 μm nozzle (Anest Iwata, Yokohama) modified to run continuously. The airbrush consisted of an inner feed line, fed by gravity, leading to a nozzle and an outer air line, ending in a cap to form a coaxial outlet with the nozzle (FIG. 1A and FIG. 1B). The low pressure created by the escaping air drew solution from the inner nozzle. Air pressure on the air brush was varied from 1 psi to greater than 30 psi. Air pressure was set to 30 psi for scaffolds produced in various embodiments.

A range of collagen solution concentrations between 5 % and 10 % wt/vol was tested to identify the optimal solution that could be reliably dispensed. Solutions below 6 % formed insufficient fibers, while those above 9 % tended to clog the nozzle of the airbrush. A 9 % solution of collagen was chosen for subsequent production of scaffolds. 60 mL of collagen solution generated a 1 mm thick scaffold of collagen fibers in less than 2 hours. This was significantly faster than electrospinning experiments which required over 24 hours to generate 250 μm thick scaffolds.

During production of pneumatospun collagen scaffolds, the collector mesh was rotated at constant speeds from 10 rpm to over 650 rpm, 650 rpm was chosen as an ideal constant rate of rotation for scaffold collection. Rotation speeds were also varied within this range of 10 rpm to over 650 rpm to determine the effect of varying the speed on the characteristics of created scaffolds.

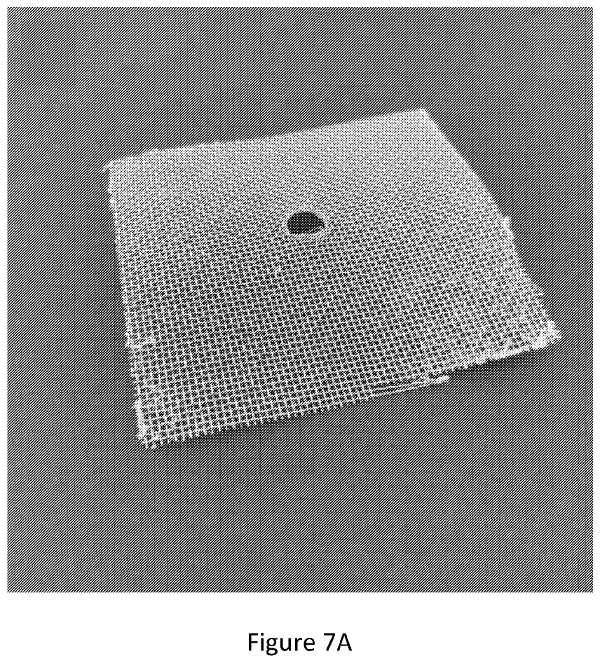

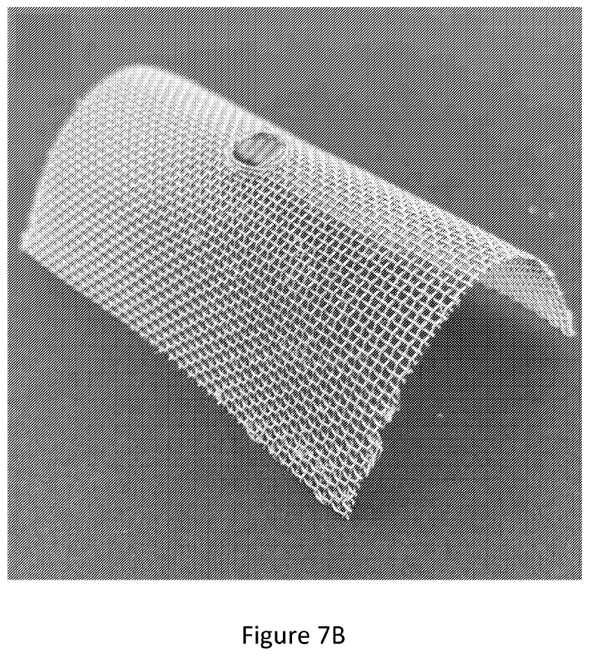

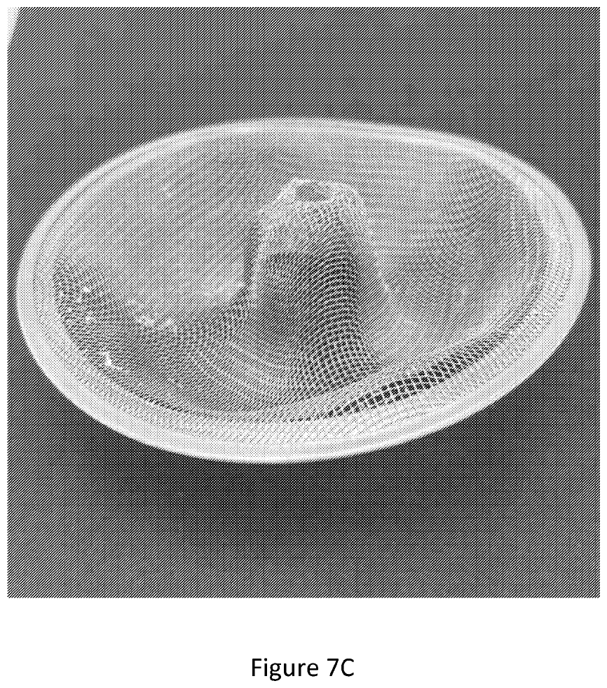

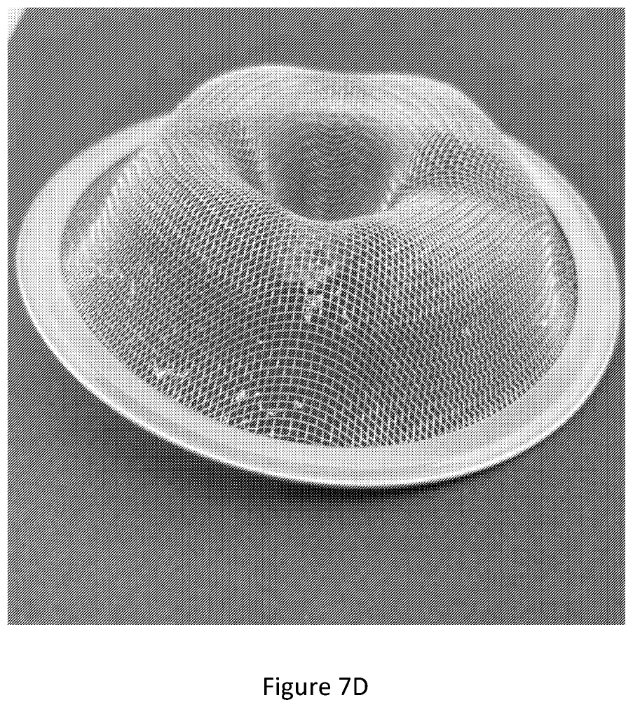

Flat square mesh collectors (FIG. 7A) were used to create scaffolds which were then further cut and shaped into various formats including that of a meniscus. The potential to create scaffolds of varying shapes was demonstrated by using curved targets with radial ripples (FIG. 7B-7D). Glutaraldehyde crosslinked mats spun onto these targets retained the curvature of the targets after removal, but showed no appreciable variation in thickness. Liquid or vapor glutaraldehyde may be used.

### Example 2: Scaffold Properties

Before crosslinking, spun collagen mats were fragile and could fragment upon removal from the target substrate. They also could dissolve in culture. After glutaraldehyde crosslinking, collagen mats could be easily removed intact from the target. By 3 weeks, cultured scaffolds swelled and softened, but retained their net spun shape and could be easily manipulated with forceps for loading into mechanical testing fixtures or for surgical implantation in ex vivo tissue samples.

Collagen scaffolds were subjected to glutaraldehyde (GA, 25%, Thermo Fisher Scientific, Waltham, MA) vapor to induce crosslinking. Collagen scaffolds on the collector mesh were suspended over an open beaker containing 30 mL GA solution and enclosed in a glass container for 48 hours to contain the GA fumes in a chemical fume cabinet at 40° C. After glutaraldehyde crosslinking, scaffolds were cut to size and washed in PBS four to five times and stored in PBS at 4° C.

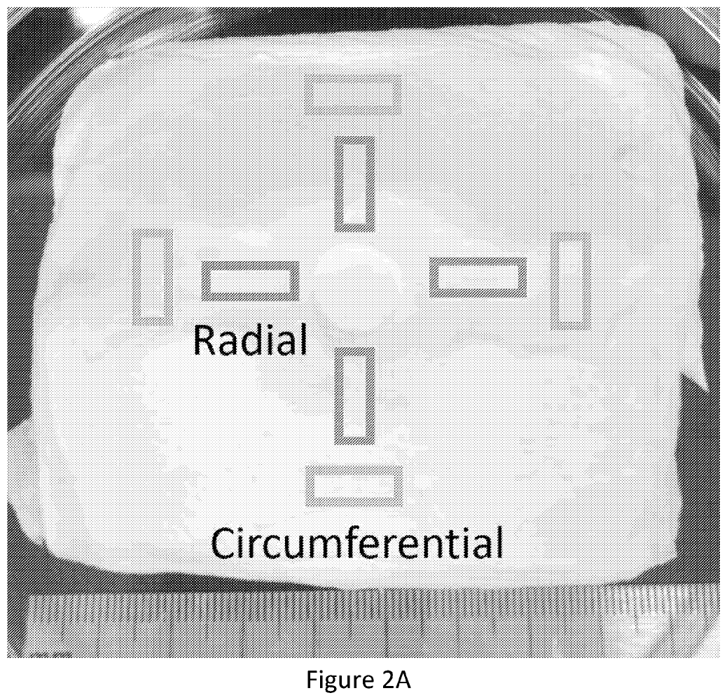

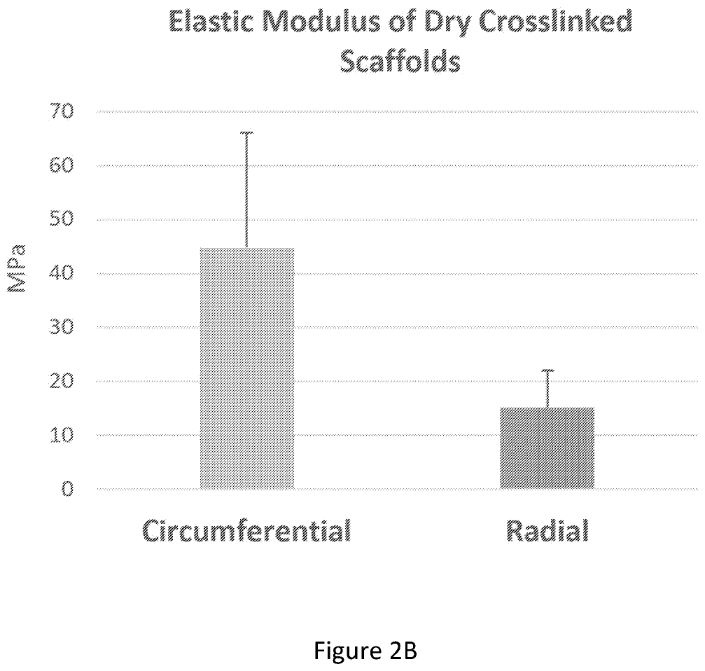

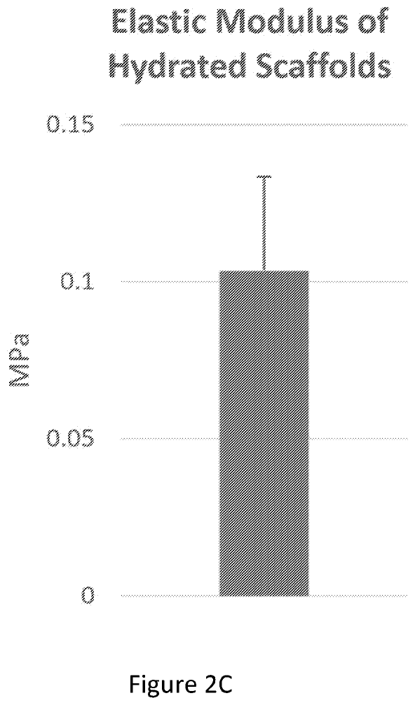

Scaffold specimens were tested for tensile strength and tensile modulus using an Instron 8511 servohydraulic testing machine (Instron, Norwood, MA). Briefly, rectangular sections of 8 mm by 26 mm were cut, some with the long axis tangent to the rotation of the collector (circumferentially oriented) and some with the long axis radial to the rotation of the collector (radially oriented, FIG. 1A and FIG. 2A). Specimens were mounted with a set of Instron grips and loaded to failure under tension at 4.2 mm/s. Load and displacement were recorded throughout the loading process.

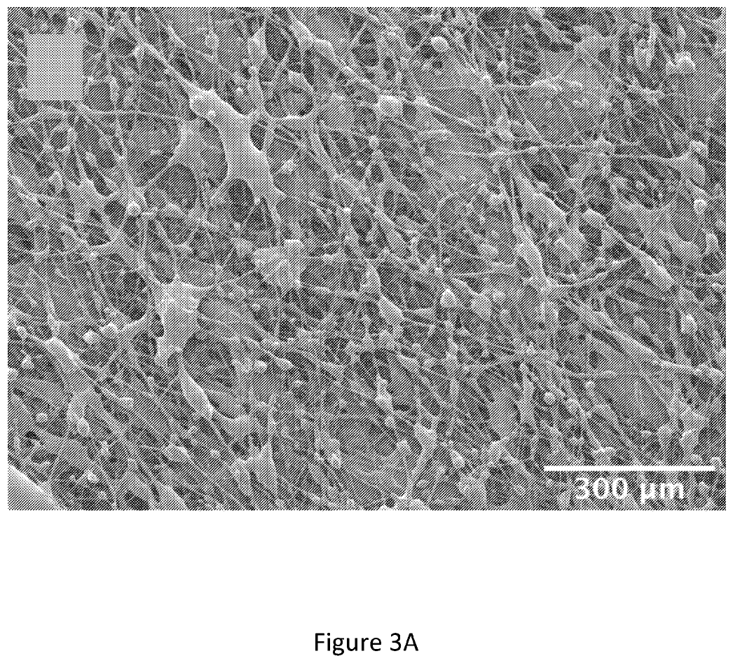

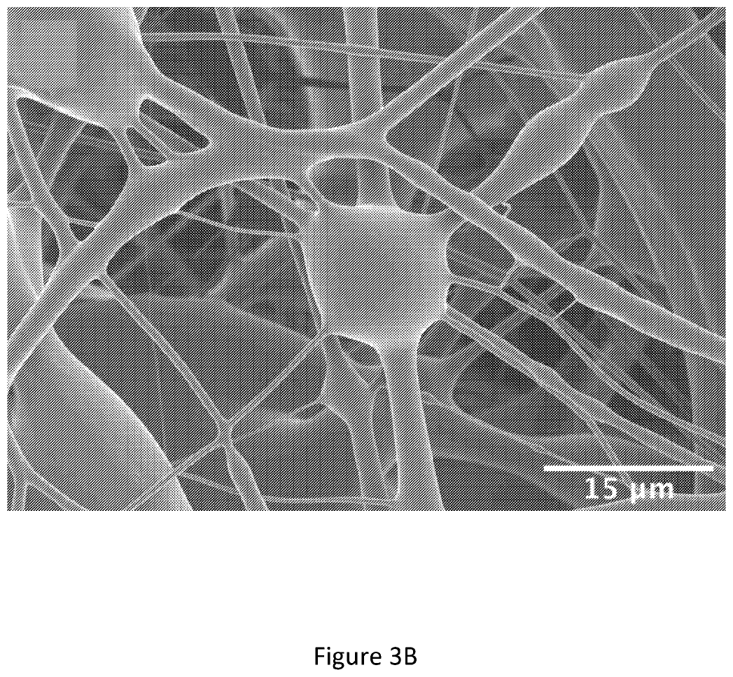

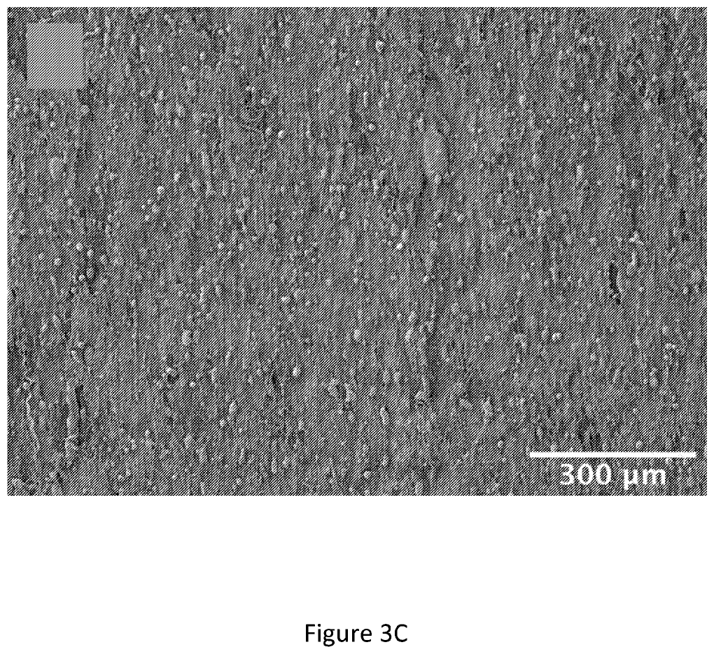

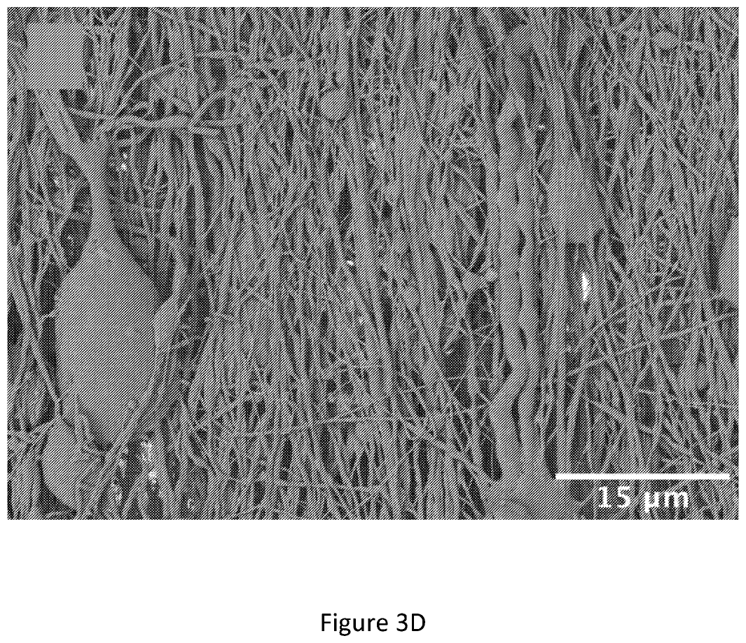

Dry collagen GA crosslinked scaffolds exhibited an clastic modulus of around 45 MPa, which was reduced significantly after hydration to 0.1±0.03 MPa (FIG. 2B-2C). Dry scaffolds failed consistently within the gauge length of the test setup, while hydrated scaffolds tended to fail near the tissue grips. Scanning electron microscopy (SEM) imaging of the GA crosslinked pneumatospun scaffolds revealed a heterogenous distribution of fiber sizes and large, interconnected porosities (FIG. 3A-3B). This contrasted with aligned, electrospun scaffolds which have low porosity and more uniform, nanoscale fibers (FIG. 3C-D). For SEM, scaffolds were coated with 3.7 nm of Iridium and imaged on a Hitachi S-4800 SEM at 3 kV and 8 mm WD. SEM images were assessed for porosity and fiber morphology using ImageJ software (NIH, Bethesda, MD).

Pneumatospun fibers appeared irregular with diameters ranging from 1 μm to 30 μm. The overall porosity of the collagen scaffold was approximately 48% with pore sizes ranging from 7.4 μm to 100.7 μm with a median of 23.8 μm and a mean of 25.7 μm. We also observed bead formation which is related to the surface tension of the solution and can be reduced by optimizing solution and spinning parameters. Porosity was measured using ASTM F1854-15 (ASTM, 2015). Briefly, images were segmented to separate foreground fibers from background. Four lines were drawn horizontally across the image and values were measured along the corresponding profile. The percentage of pore space along these profiles was averaged to arrive at a value for the image. Pore size was measured across the longest axis of the pores.

### Example 3: Neotissue Formation

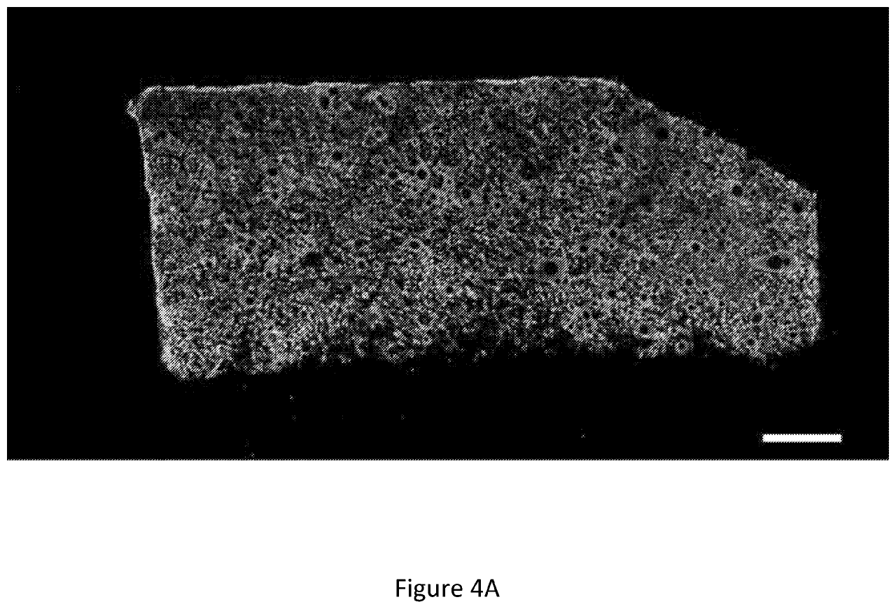

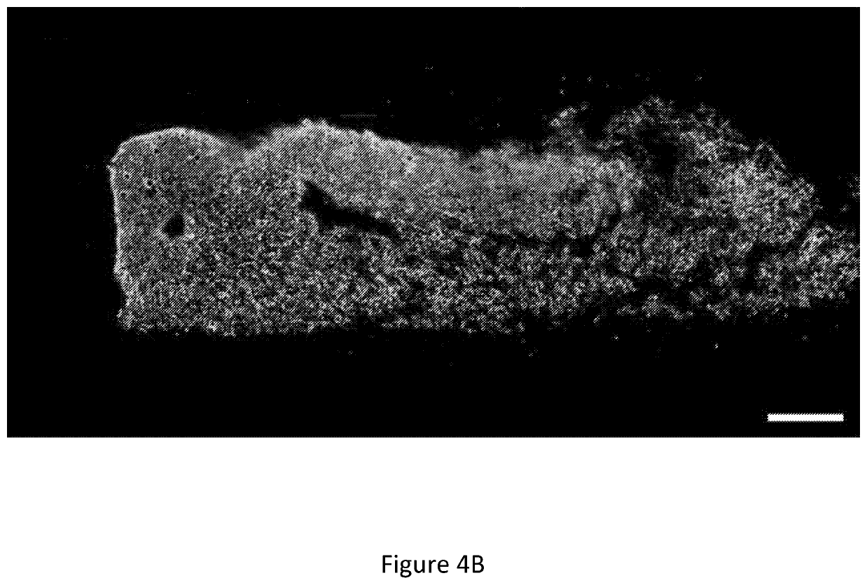

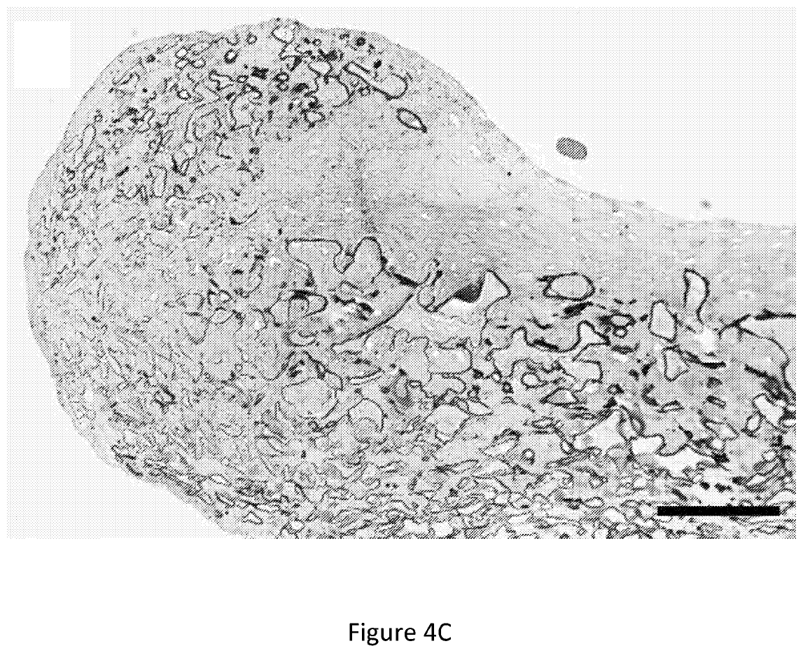

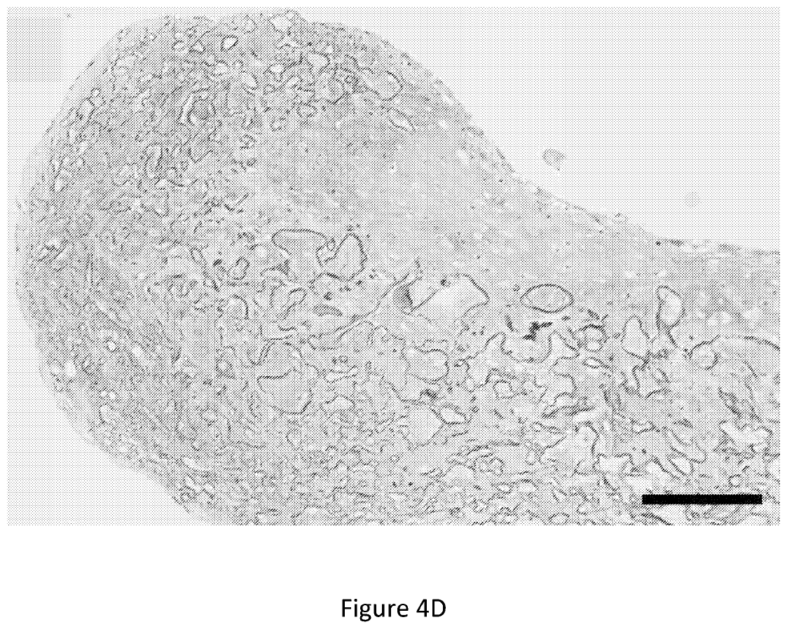

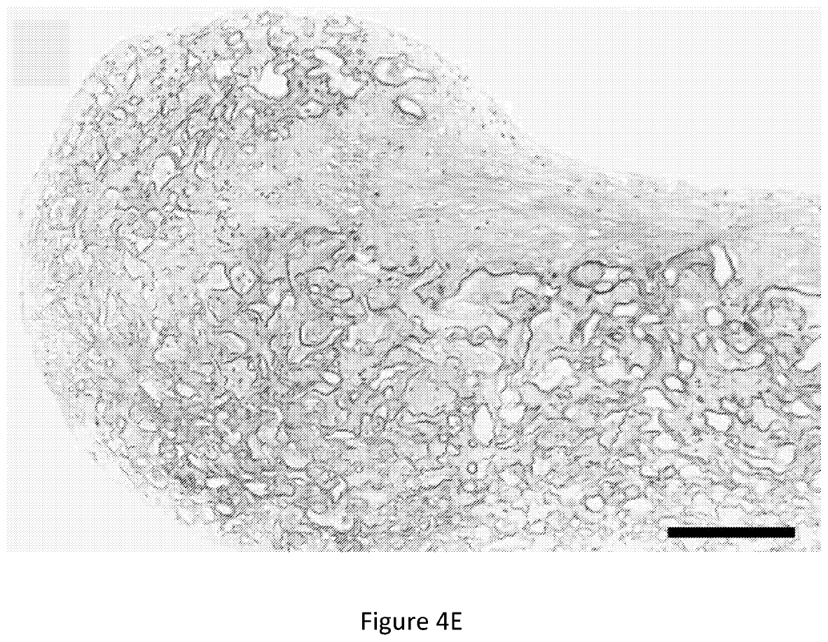

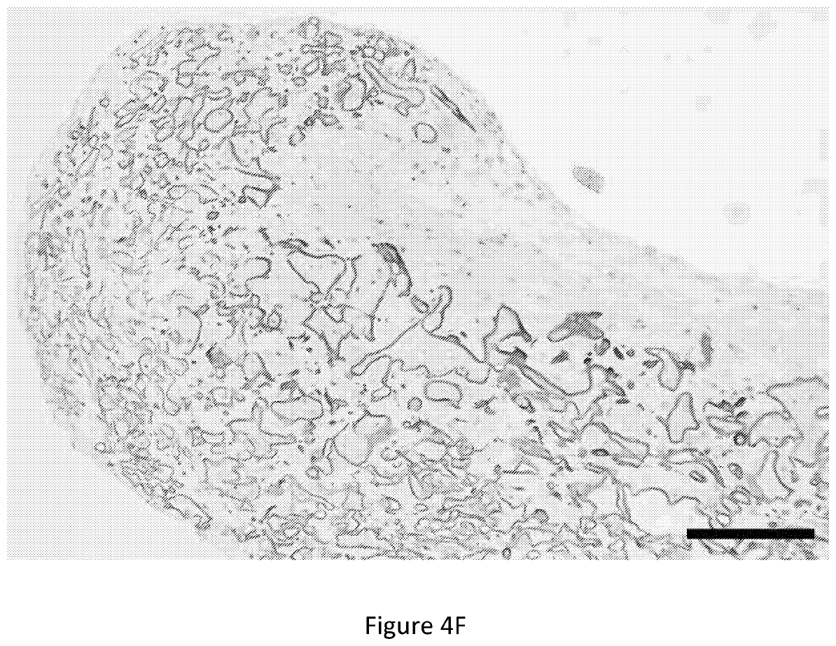

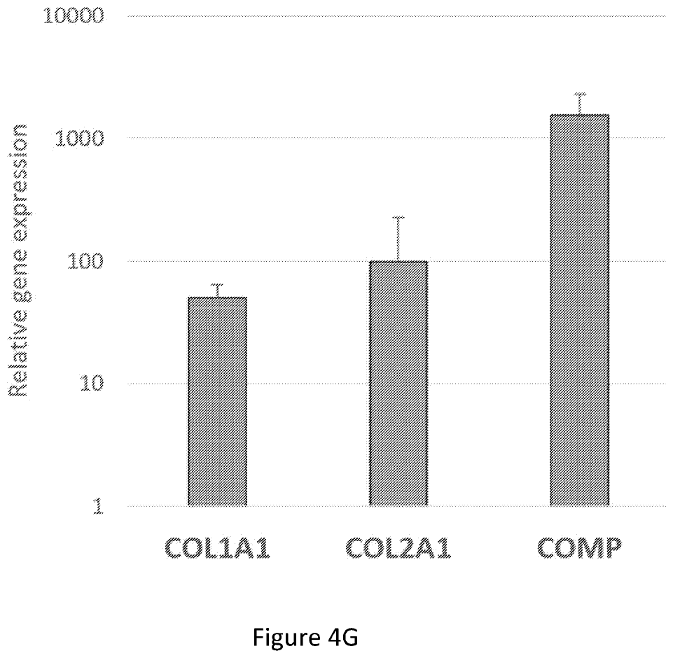

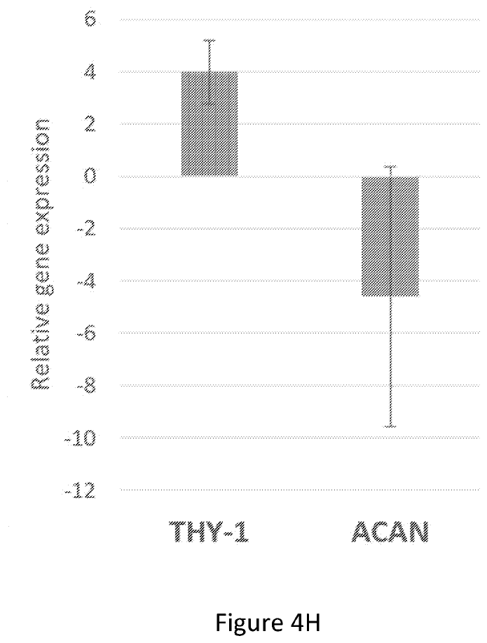

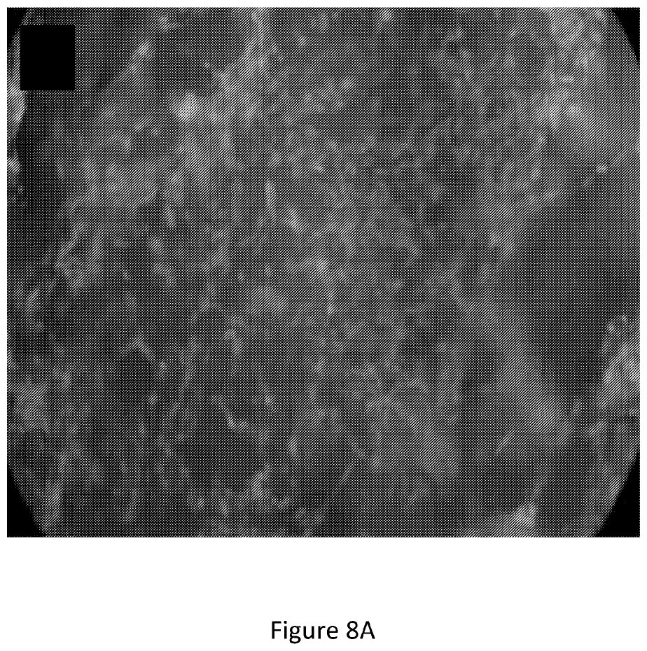

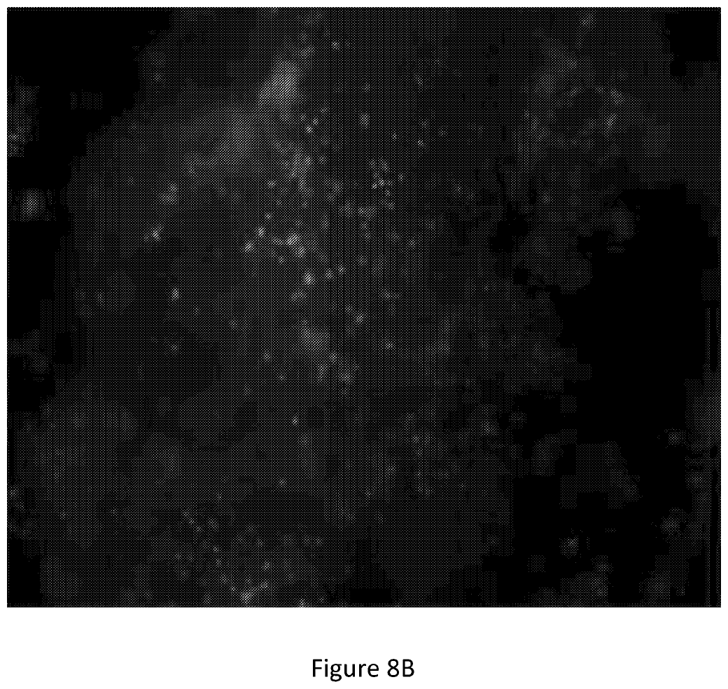

Neotissue formation was assessed in pneumatospun scaffolds. Glutaraldehyde crosslinked pneumatospun collagen scaffolds were seeded with IPFP-MSC, cultured in 6-well plates in MSC medium and placed on an orbital shaker overnight in the incubator. This approach enhanced cell attachment and infiltration into the scaffold (FIG. 8A-8B). After 3 weeks of culture in serum-free differentiation medium. IPFP-MSC were observed throughout the pneumatospun collagen scaffolds with 70.2±7.5% viability (FIG. 4A-B).

Infrapatellar fat pad (IPFP) tissue was obtained from patients (74 year old female and 75 year old male) undergoing total knee replacement (approved by Scripps Institutional Review Board). Mesenchymal stem cells (IPFP-MSC) were isolated using a previously described method (Grogan et al., 2020). Briefly, IPFP tissues were minced using a scalpel to create fragments (˜5 mm³), which were placed into 6-well plate wells precoated with human collagen type I (Cell Adhere, StemCell Technologies, Vancouver, Canada). For the first 12 hours, the tissue fragments were maintained in a CO₂ incubator at 37° C. in only 0.5 mL MSC-medium (LONZA, Walkersville, MD) supplemented with Fibroblast Growth Factor 2 (FGF-2) (10 ng/mL; PeproTech, RockyHill, NJ). After 12 hours, 1.5 mL of medium was added and the tissue fragments were cultured for 1-2 weeks until emergence of cells from the tissue. The remaining tissue fragments were discarded, and the emerging cells were detached using Accutase (Innovative Cell Technologies, Inc. San Diego, CA) and reseeded into collagen coated flasks at a density of 350,000 cells per cm².

On histological analysis, IPFP-MSC seeded into the scaffolds generated a fibrocartilage-like neotissue with extracellular matrix containing glycosaminoglycans (Safranin O positive; FIG. 4C), collagen types I and II (FIG. 4D-4F). Pneumatospun and glutaraldehyde cross-linked collagen scaffolds were cut into rectangular specimens (approx. 1 cm by 0.5 cm) and washed in PBS extensively (at least 5 times in 5 ml PBS over 24 hours). These scaffolds were seeded with IPFP-MSC (0.5×10⁶ cells per scaffold specimen), cultured in MSC medium, and placed upon an orbital shaker at 100 RPM (Ohaus Parsippany, NJ) in a CO₂ incubator at 37° C. The seeded scaffolds were maintained in MSC medium for 3-4 days for initial cell proliferation before changing to differentiation medium that consisted of Dulbecco's Modified Eagle Medium (DMEM) (Mediatech Inc., Manassas, VA), 1×ITS+1 supplement (Sigma-Aldrich, St. Louis, MO), 100 nM Dexamethasone (Sigma), 1.25 mg/ml human serum albumin (Bayer. Leverkusen, Germany), 100 μM ascorbic acid 2-phospahate (Sigma), 1% penicillin/streptomycin/gentamycin (PSG, Gibco, Carlsbad, CA) and supplemented with 10 ng/ml TGFβ3 (PeproTech, Rocky Hill, NJ). Scaffolds were cultured for 3 weeks (with medium changes every 3-4 days) to assess cell attachment, viability, and gene expression.

Cell viability and distribution after 24 hours or following 3 weeks in culture was measured with the Live/Dead kit (Invitrogen, Waltham, MA). The staining buffer consisted of Ethidium Homodimer-1 (8 mM) and Calcein-AM (1.6 mM) suspended in PBS. The cell seeded collagen scaffolds were incubated for 30-40 min before visualization with either a fluorescence microscope (Axiovert 200M, Zeiss, Jena, Germany) or via a confocal laser microscope (LSM-810; Zeiss). The percentage of live and dead cells was calculated using ImageJ/Fiji.

Histology was used to characterize new tissue growth. To assess histology, extend and characteristics of new tissue growth. Paraffin embedded sections (4 μm thick) were mounted on glass slides for staining with Safranin O-fast green or immunohistochemistry for collagen types I and II as previously described. Briefly, paraffin cut sections were deparaffinized and treated with pepsin for 9 minutes at 37° C. in a humidified chamber (Digest-All 3, Thermo Fisher Scientific). Rabbit anti-human collagen type I antibody (1 μg/ml; Ab 34710, Abcam, Cambridge, MA) and mouse anti-human collagen type II (2 μg/ml; II-II6B3, Hybridoma Bank, University of Iowa) was used as the primary antibodies, which were incubated at 4° C. for 12 to 16 hours in a humid chamber. The ImmPRESS secondary DAB (Brown) or AP (red) kits were used for color development (Vector Laboratories, Burlingame, CA). Non-specific staining was evaluated using species-matched isotype controls at the same concentration as the specific primary antibodies.

This histology was generally consistent with the gene expression profile of increased COL1A1, COL2A1 and COMP, THY-1 (CD90) and reduced ACAN expression compared to IPFP-MSC in monolayer culture (FIG. 4G-4H). To assess gene expression following the manufacturer's instructions (Qiagen, Valencia, CA), scaffolds were minced by scalpel in RLT buffer and then subjected to homogenization using the Qiashredder. Total RNA was extracted using the RNeasy kit (Qiagen) and cDNA was made using the High Capacity cDNA Reverse Transcription Kit (Applied Biosystems, Foster City, California). Verified primer/probe assays were purchased from Applied Biosystems to monitor the expression levels of COL1A1, COL2A1, COMP, ACAN, and THY1 relative to the GAPDH (housekeeping gene). The gene expression levels of IPFP-MSC in monolayer culture were used to calculate relative changes in expression level following culture on the pneumatospun collagen scaffolds.

### Example 4: Repair of Ex Vivo Meniscus Tears

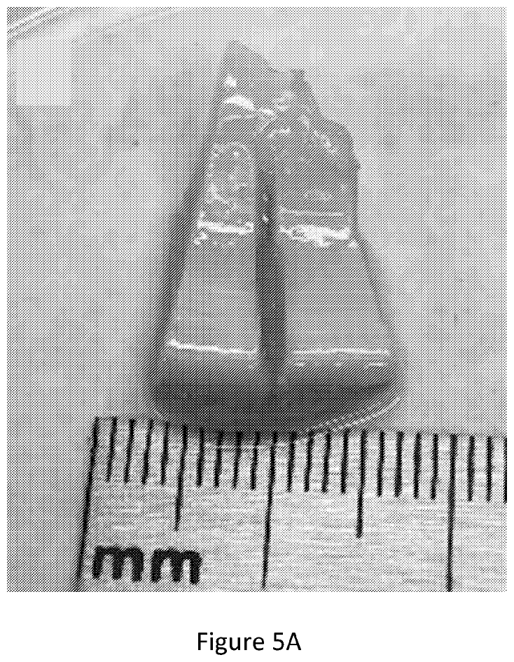

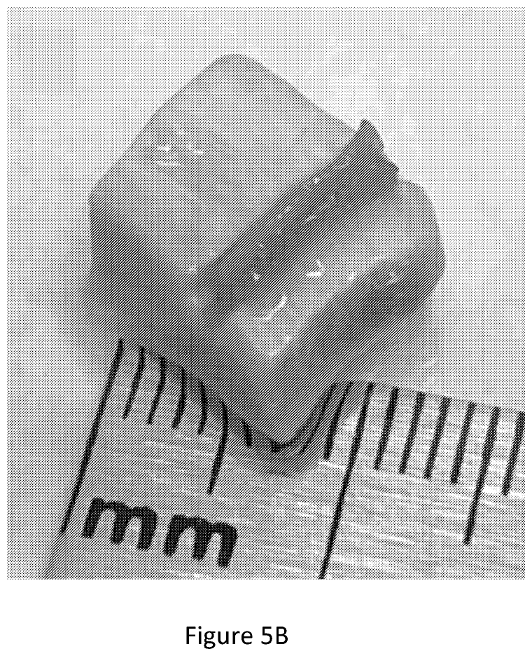

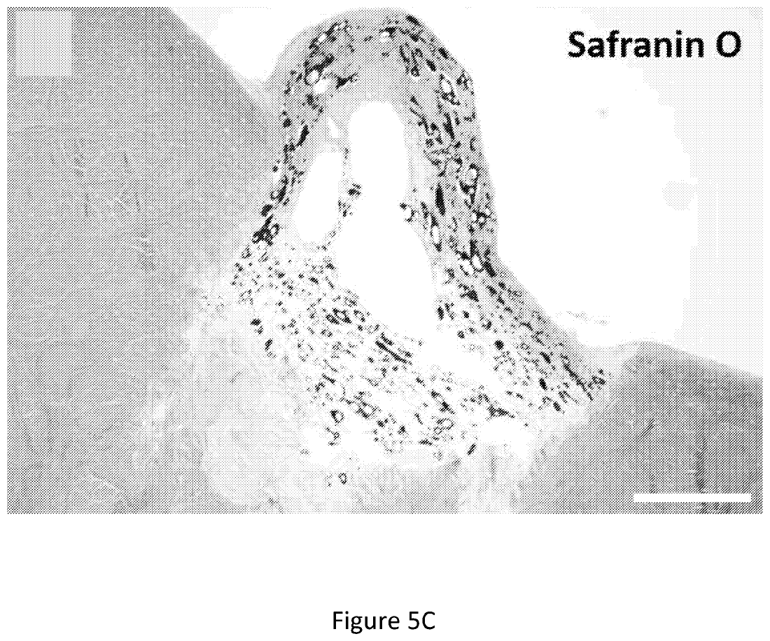

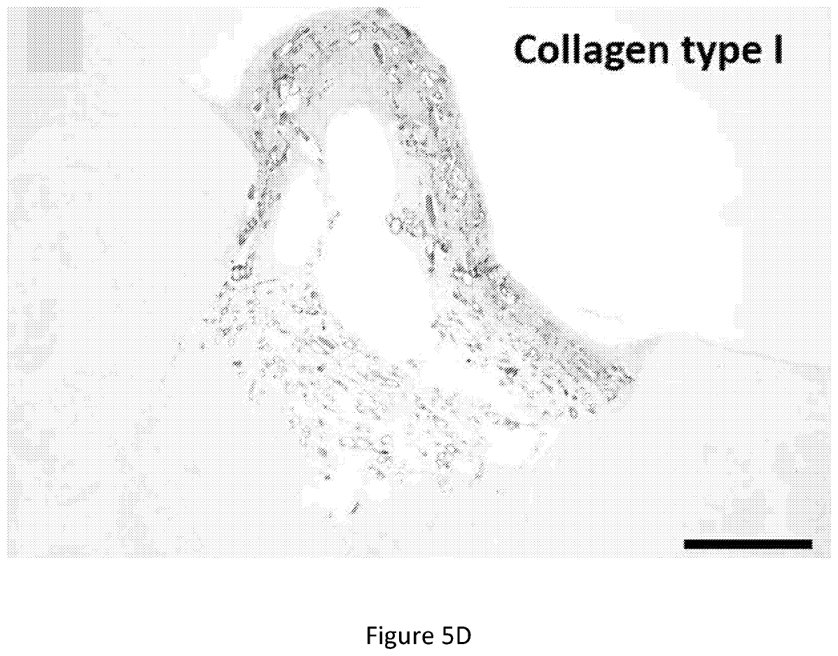

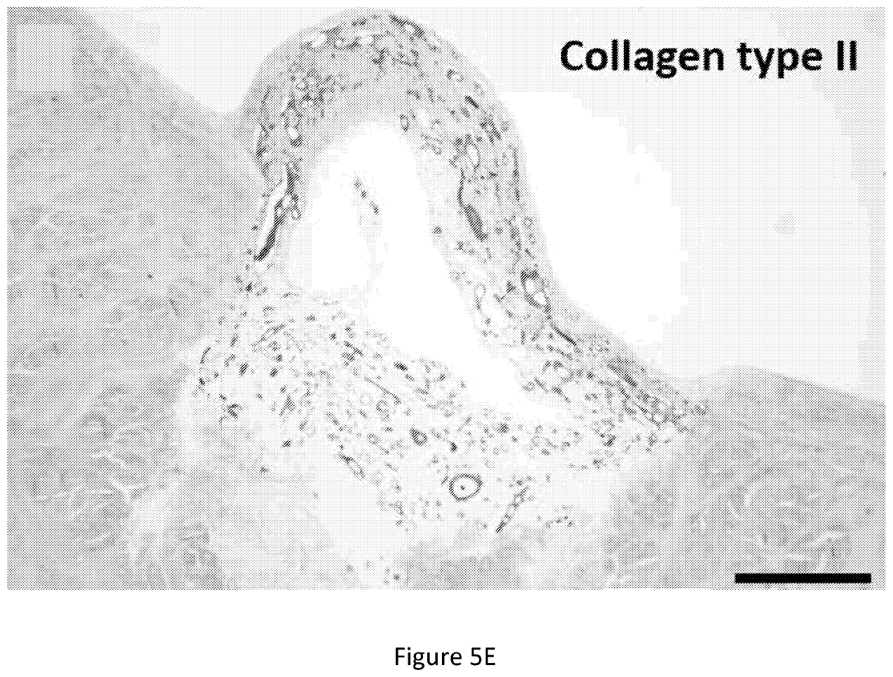

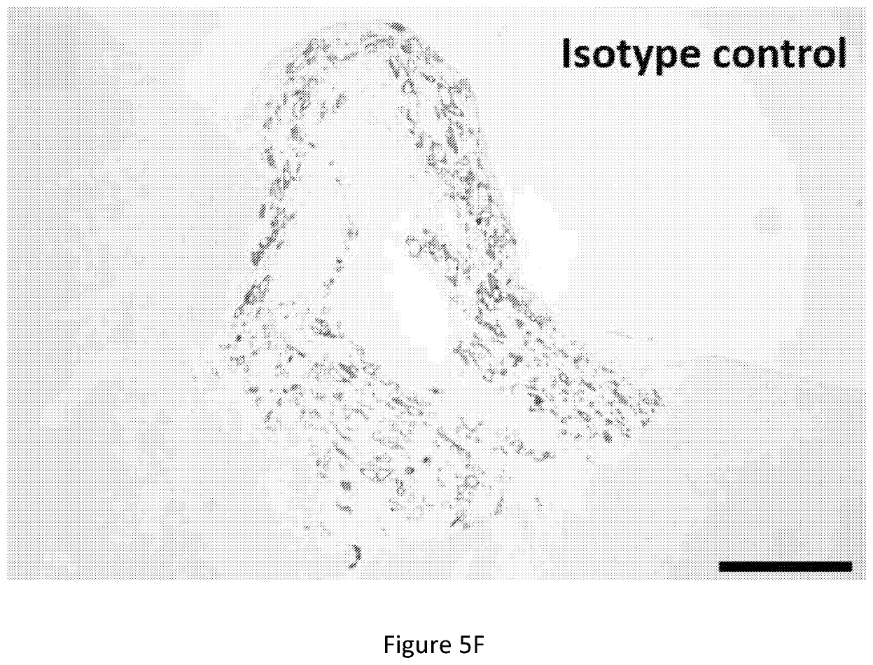

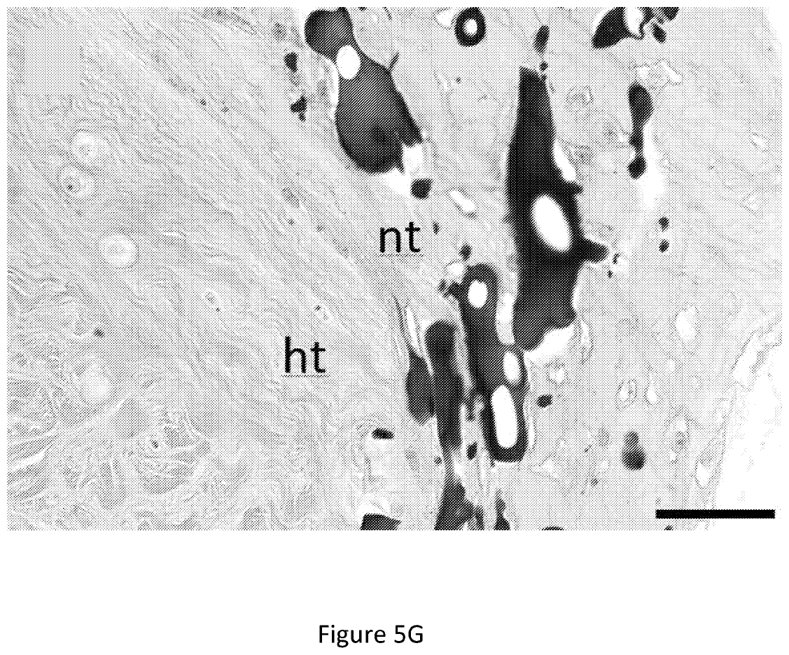

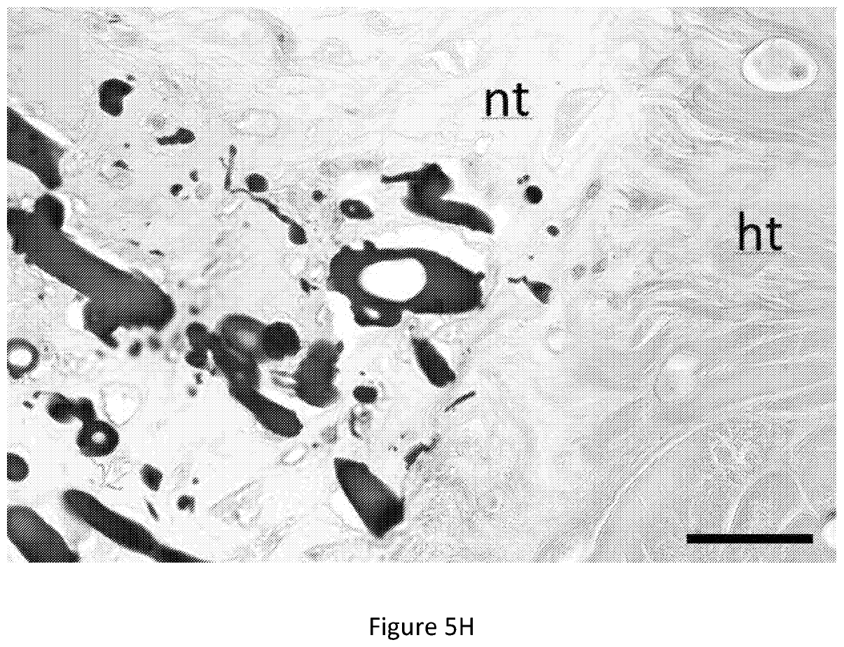

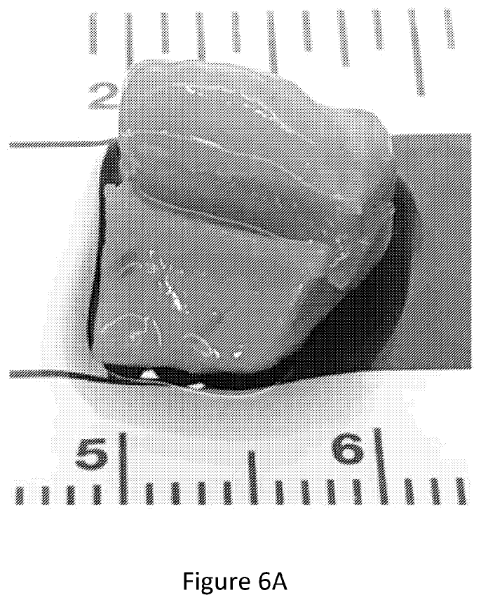

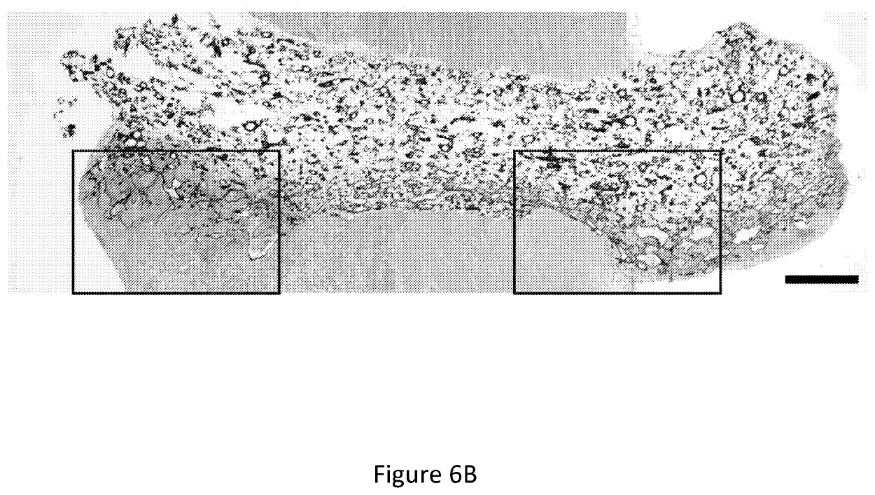

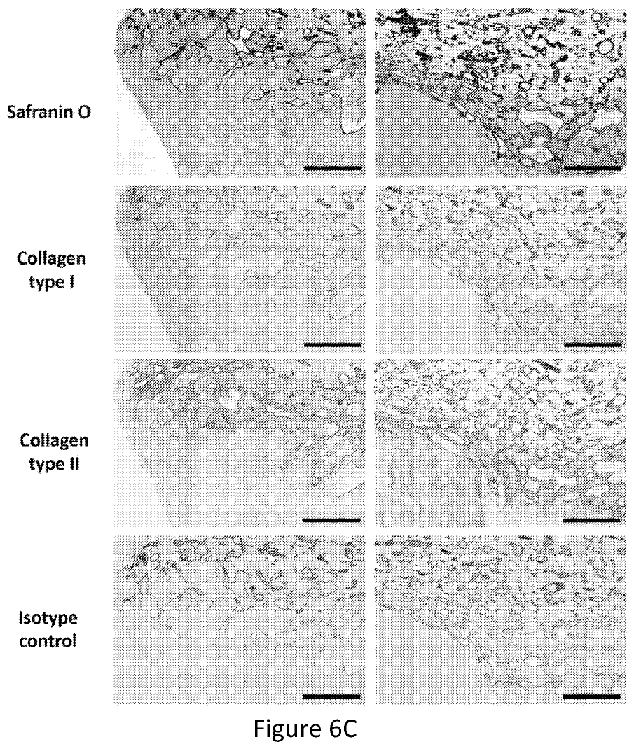

In another example described herein, the potential for repair of meniscus tears was assessed by implanting scaffolds seeded with IPFP-MSC into surgically created defects in normal healthy bovine meniscus (FIG. 5A-5B) or in osteoarthritic human meniscus resected from patients that had undergone TKA (FIG. 6A). An ex vivo culture system was used to assess repair of meniscus tears.

To create an ex vivo meniscus repair model, whole bovine knees with the knee capsule intact were obtained from Animal Technologies, Inc. (Tyler, TX). Meniscal tissue explants of approximately 1 cm wide and×3 cm deep were cut under sterile conditions. Explants were cultured in DMEM with 10% CS and 1% PSG until ready to use. Collagen scaffolds (approx. 1 cm long, 0.5 cm wide, and 0.2 cm thick) produced by pneumatospinning were seeded with IPFP-MSC (0.5×10⁶ per scaffold) and cultured for 3 days in MSC-medium, followed by 7 days in differentiation medium. Scaffolds were implanted to repair surgically created longitudinal or transverse defects in bovine explants. The implanted scaffolds and explant tissue were cultured for 3 weeks in the differentiation medium, with changes every 3-4 days. At the end of culture, the explants were fixed and processed for paraffin embedding and subsequent histological analyses.

To create an ex vivo meniscus repair model using human tissue, menisci were obtained from four patients (69.3±10.1 years, three female, one male) following total knee arthroplasty (approved by Scripps Institutional Review Board). The menisci were cut into tissue explants of around 1 cm×1 cm and each cultured in DMEM with 10% CS and 1% PSG until ready for scaffold implantation (within 2 days). Collagen scaffolds (approx. 1 cm long, 0.5 cm wide, and 0.2 cm thick) produced by pneumatospinning were seeded with IPFP-MSC (0.5×10⁶ per scaffold) and cultured for 3 days in MSC-medium, followed by 7 days in differentiation medium. Scaffolds were implanted to repair surgically created longitudinal or transverse defects in human explants. The implanted scaffolds and explant tissue were cultured for 3 weeks in the differentiation medium, with changes every 3-4 days. At the end of culture, the explants were fixed and processed for paraffin embedding and subsequent histological analyses.

After 3 weeks of culture in differentiation medium, neo-fibrocartilaginous tissue developed in the bovine meniscus that integrated with the host tissue (FIG. 5). More GAG deposition was noted in the superficial region corresponding spatially with the GAG seen in the host tissue via Safranin O staining (FIG. 5C). Collagen type I and Collagen type II protein expression in the cultured bovine explants was also assessed against an isotype control (FIGS. 5D-5F). The mix of collagen types I and II indicated a fibrocartilage-like composition. Higher magnification of Safranin O stained image showing integration of neotissue (nt) with host tissue (ht). The collagen scaffold is stained dark blue (FIGS. 5G-5H).

Cell-seeded collagen scaffolds implanted into osteoarthritic human menisci (FIG. 6A) also resulted in similar fibrocartilage-like neotissue which integrated with host tissue with a positive Safranin O and a collagen type I and II staining profile (FIG. 6B and 6C).

In both bovine and human explants, histology and immunohistochemistry was conducted using the following protocols. Paraffin embedded sections (4 μm thick) were mounted on glass slides for staining with Safranin O-fast green or immunohistochemistry for collagen types I and II as previously described. Briefly, paraffin cut sections were deparaffinized and treated with pepsin for 9 minutes at 37° C. in a humidified chamber (Digest-All 3, Thermo Fisher Scientific). Rabbit anti-human collagen type I antibody (1 μg/ml; Ab 34710, Abcam, Cambridge, MA) and mouse anti-human collagen type II (2 μg/ml; II-II6B3, Hybridoma Bank, University of Iowa) was used as the primary antibodies, which were incubated at 4° C. for 12 to 16 hours in a humid chamber. The ImmPRESS secondary DAB (Brown) or AP (red) kits were used for color development (Vector Laboratories, Burlingame, CA). Non-specific staining was evaluated using species-matched isotype controls at the same concentration as the specific primary antibodies.

### Example 5: Pneumatospinning of Collagen Type I Scaffolds

In this experiment, collagen type I scaffolds were fabricated by pneumatospinning. The protocols used in this experiment are similar to the protocols described in Dorthé et al. 2022 (PMID: 35186903). Collagen type I was dissolved in HFIP at a concentration of 9% wt/vol to establish a concentration for spinning. This resulted in a collagen solution. The collagen solution was loaded into a syringe that was mounted on a syringe pump. Alternatively, the collagen solution was gravity fed to a gravity fed air brush with a 350 μm nozzle (Anest Iwata, Yokohama) set to run continuously. The air brush consisted of an inner solution feed line, leading to a nozzle and an outer channel, and ending in a nozzle oriented coaxially around the solution outlet. The low pressure created by the escaping air drew the solution from the inner nozzle. The air pressure on the air brush was set to 30 psi. FIGS. 1A and 1B show an example pneumatospinning setup.

A square section of approximately 90 mm×90 mm of flat stainless-steel mesh was used as the target (with a 0.012″ wire diameter; TWP Inc, Berkeley). The target was mounted on an electric motor and rotated at 650 RPM with the axis of rotation co-linear with the axis of the airbrush nozzle. The distance between the nozzle and target was set to 12 cm.

A total of 100 ml of the collagen/HFIP solution was used to produce a random nanofiber collagen scaffold that was 90 mm in width with a thickness of over 1 mm. Scaffolds were crosslinked by suspending the scaffolds over an open beaker containing 30 mL glutaraldehyde (GA) solution (Thermo Fisher Scientific, Waltham, MA) in an enclosed glass container for 48 hours to contain the GA fumes in a chemical fume cabinet at 40° C. Following crosslinking, the scaffolds were removed from the collector, cut to size, and washed in PBS four to five times. The scaffolds were stored in PBS at 4° C.

Fabrication of collagen scaffolds by pneumatospinning resulted in thicker matrices than matrices produced by electrospinning. Pneumatospinning produced the scaffolds easier and at a much faster rate than scaffolds produced by electrospinning. As a result, scaffolds with a 1 mm thickness were produced in 2 hours, which would typically take over 24 hours if done by electrospinning.

Thicknesses of up to 5 mm were observed. Scanning electron microscopy (SEM) indicated fiber diameters ranging from 1 μm to 30 μm. SEM also indicated porosity levels of approximately 48%, which exceeds the porosity levels typically produced by electrospinning. No alignment was noted in the microscopy. However, mechanical testing suggested some improvement in tensile strength between specimens spun onto a rotating target, and those on a static target.

### Example 6: Production of Composite Mats Comprised of Electrospun Aligned Nanofiber Polylactic Acid (PLA) and Random Nanofiber Pneumatospun Collagen Type I

The Spinbox electrospinning system was used for electrospinning polylactic acid (PLA) (Nanoscience Instruments, Phoenix, AZ). PLA was dissolved in HFIP at a concentration of 20% and was loaded into a syringe mounted on a syringe pump. The PLA/HFIP solution was delivered at a rate of 2 ml/hr to a nozzle (21 gauge). A voltage was applied to the nozzle. The voltage applied varied from 12 kilovolts (kV) to 14 kV. The distance between the nozzle and the rotating drum collector was set at 12 cm. The collector was covered in a conducting plastic. The collector was set to rotate at 2000 RPM. Active electrospinning occurred for 2 hours to produce a mat containing a thickness of 0.35 mm.

A composite PLA/collagen mat was produced. To do this, the electrospun aligned PLA scaffolds were removed from the drum collector, cut to size (approximately 90 mm×90 mm), and mounted upon a flat stainless-steel mesh collector. The collagen/HFIP solution was fed though a 350 μm nozzle with a constant air pressure of 30 psi. The collector containing the base PLA mat did not rotate for this protocol.

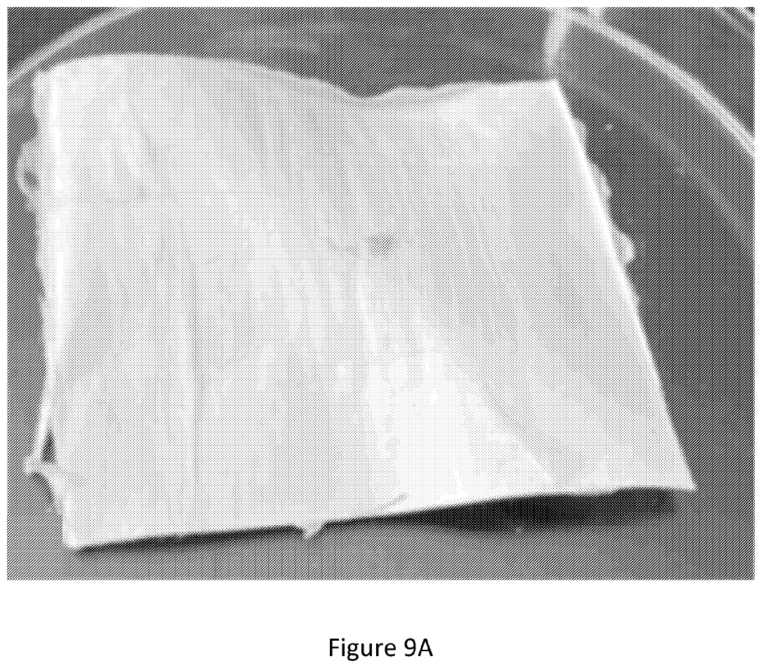

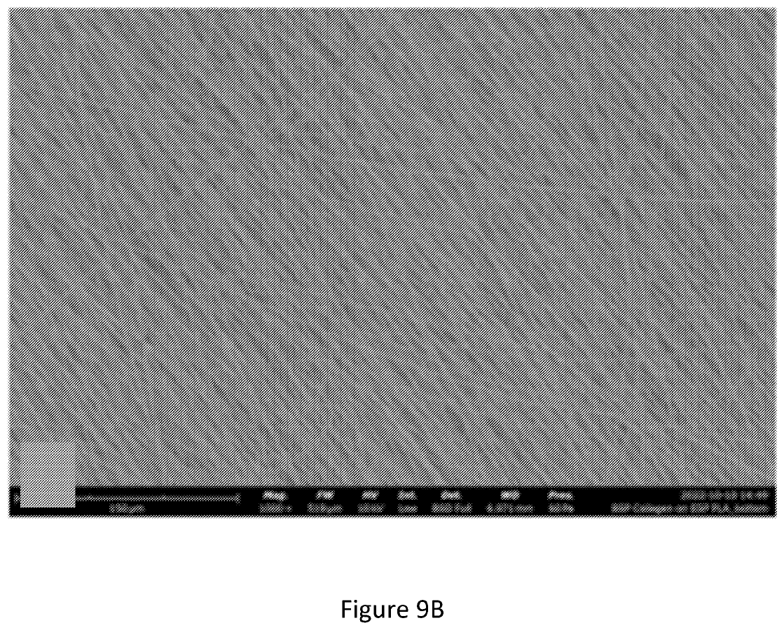

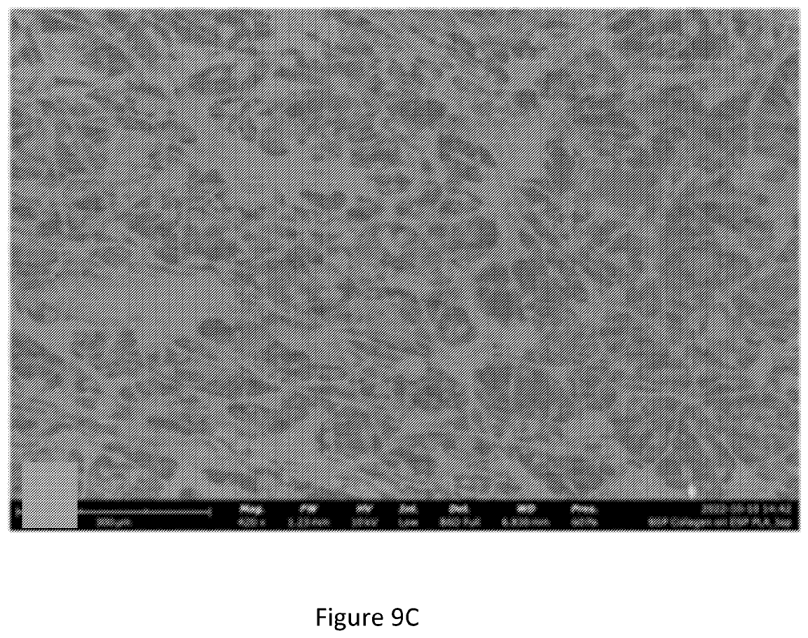

Composite maps were produced through the combination of (1) producing aligned PLA scaffolds for scaffold mechanical strength and (2) layering pneumatospun collagen type I over the PLA base. This combination provided a more biocompatible environment for cells. To demonstrate the combination, a 1.1 mm layer of collagen was pneumatospun over the base PLA. resulting in a composite mat of electrospun PLA and pneumatospun collagen (FIG. 9A). SEM images of the bottom layer comprising aligned PLA mat fibers (FIG. 9B) and the top layer comprising aligned pneumatospun collagen mat (FIG. 9C) were produced. Additional collagen may be added. Further, the scaffold may be flipped to produce two outer collagen layers and an inner PLA core mat.

### Example 7: Culture and Differentiation of Embryonic-Derived Mesenchymal Stem Cells (ES-MSC) on Pneumatospun Collagen Type I Scaffolds

Embryonic-derived mesenchymal stem cells (ES-MSC) were expanded on fibronectin coated tissue culture flasks (10 ug/ml) with serum-free growth medium (StemPro-34 serum free medium; ThermoFisher Scientific) and supplemented with 2 mM L-glutamine (ThermoFisher Scientific), 50 μg/ml Ascorbic acid (Stemcell Technologies, Vancouver, Canada), 1.8 μl/ml ITS-G (100× Insulin-Transferrin-Selenium; ThermoFisher Scientific) and bFGF 20 ng/ml (ThermoFisher Scientific).

GA crosslinked pneumatospun collagen type I scaffolds were subjected to heparin crosslinking using EDC/NHS chemistry (Lee et al. 2012 PMID: 22770570). All scaffolds were coated in fibronectin (10 μg/ml in HBSS) for 2 hours. Individual scaffolds were soaked in either 100 ng/ml of TGFμ1 or PDGFbb in HBSS overnight for conjugation. Scaffolds were rinsed with HBSS.

Three conditions were tested: i) control non growth factor treated, ii) TGFβ1 conjugated, and iii) PDGFbb conjugated. Each scaffold comprised of 4 mm by 2 mm thick discs. For each scaffold, a total of 0.1×10\hat{ }6 cells were seeded with growth medium and cultured for 7 days to permit cell infiltration. After 7 days, the medium was changed to serum-free differentiation medium (see Grogan et al. 2020 PMID: 31134817). Some scaffolds were maintained in culture wells for 4-weeks or implanted into ex vivo human meniscus tissue. The medium was changed every 3 to 4 days.

After culture, some of the scaffolds were used for histology analysis. For histology, the scaffolds were fixed in Z-Fix and processed for paraffin embedding. Sections of scaffold were mounted on glass slides and stained for Safranin O-fast green to examine the presence of glycosaminoglycans (GAG) or processed for immunohistochemistry for the presence of collage type I.

Another portion of the scaffolds were used for gene expression analysis. Scaffolds preserved for gene expression involved use of an RNA extraction kit (RNeasy, Qiagen). The total RNA was converted to cDNA (High Capacity cDNA Reverse Transcription Kit; Applied Biosystems, Foster City, CA) and gene expression analysis was performed using primer/probe sets (Applied Biosystems) for the following genes: GAPDH, COL1A1, COL2A1, THY-1, COMP, ACAN, CHAD, MKX and CILP. All gene expression levels were normalized to the house keeping gene GAPDH. Relative fold changes in gene expression were compared to the undifferentiated ES-MSC (monolayer controls) using the delta/delta CT method.

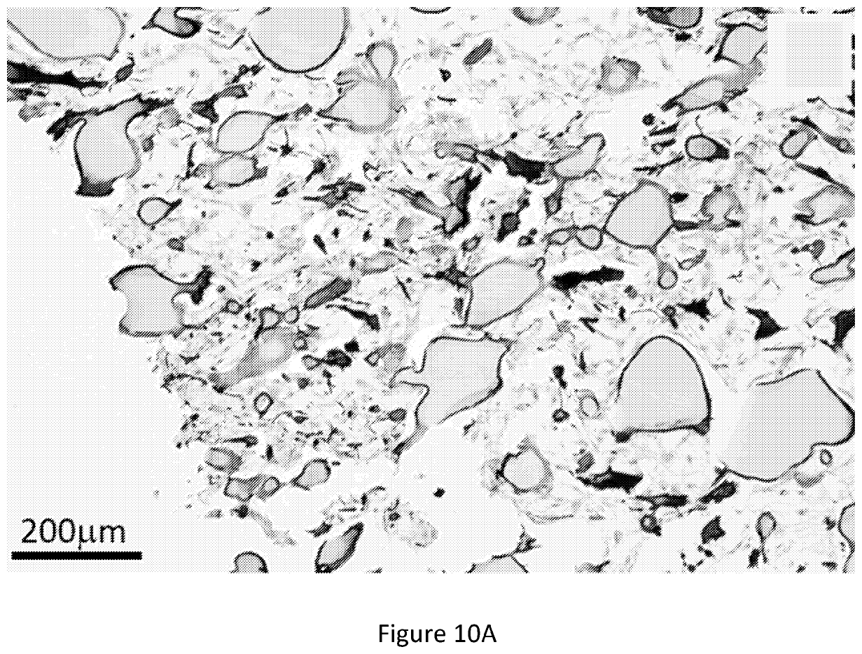

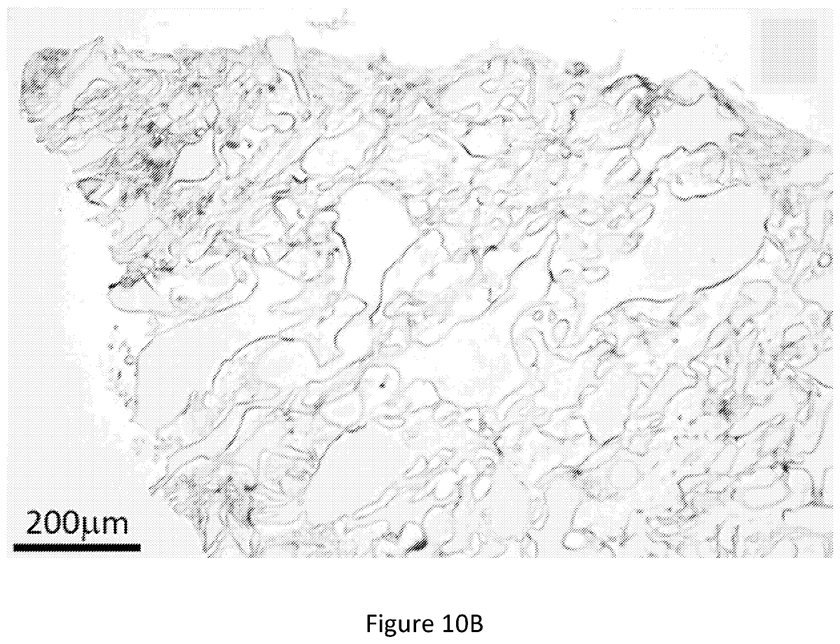

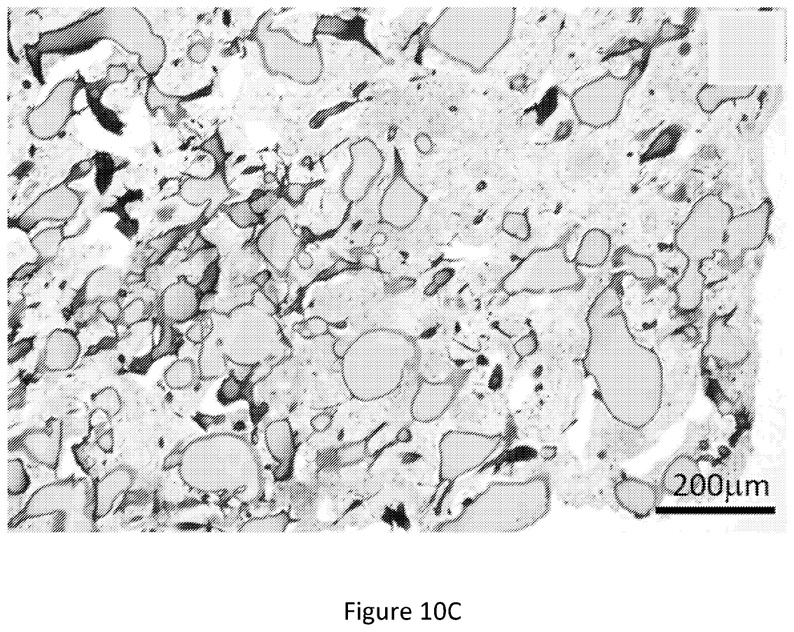

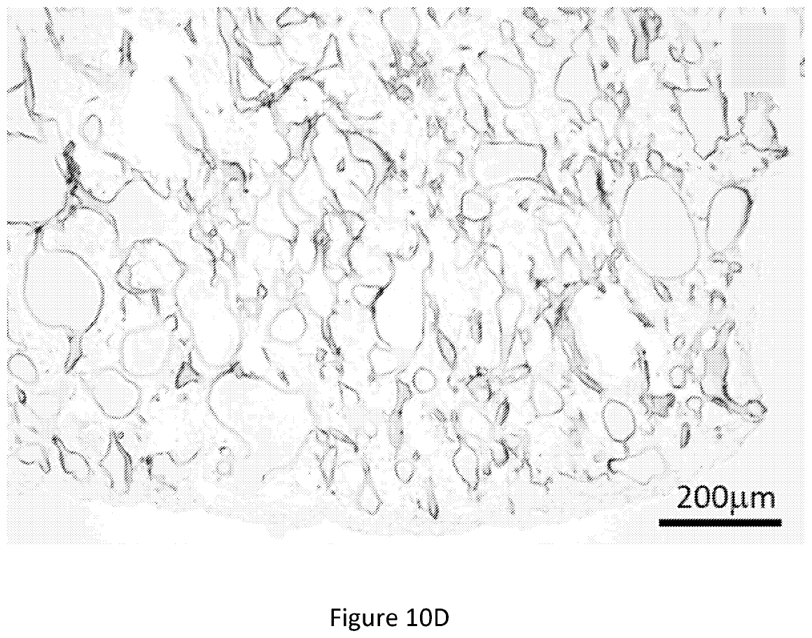

After 4 weeks of culture upon the collagen scaffolds, the ES-MSC were able to produce tissue (as shown in FIGS. 10A-10D). Uncoated control scaffolds led to the development of neo-tissues with low GAG and higher collagen type I immunostaining (FIGS. 10A and 10B), reminiscent of the vascular region of meniscus. However, ES-MSC cultivated upon heparinized and growth factor conjugated scaffolds lead to neo-tissues with higher GAG and lower collagen type I. FIGS. 10B and 10C shows cell cultivated on TGFβ1 conjugated scaffold.

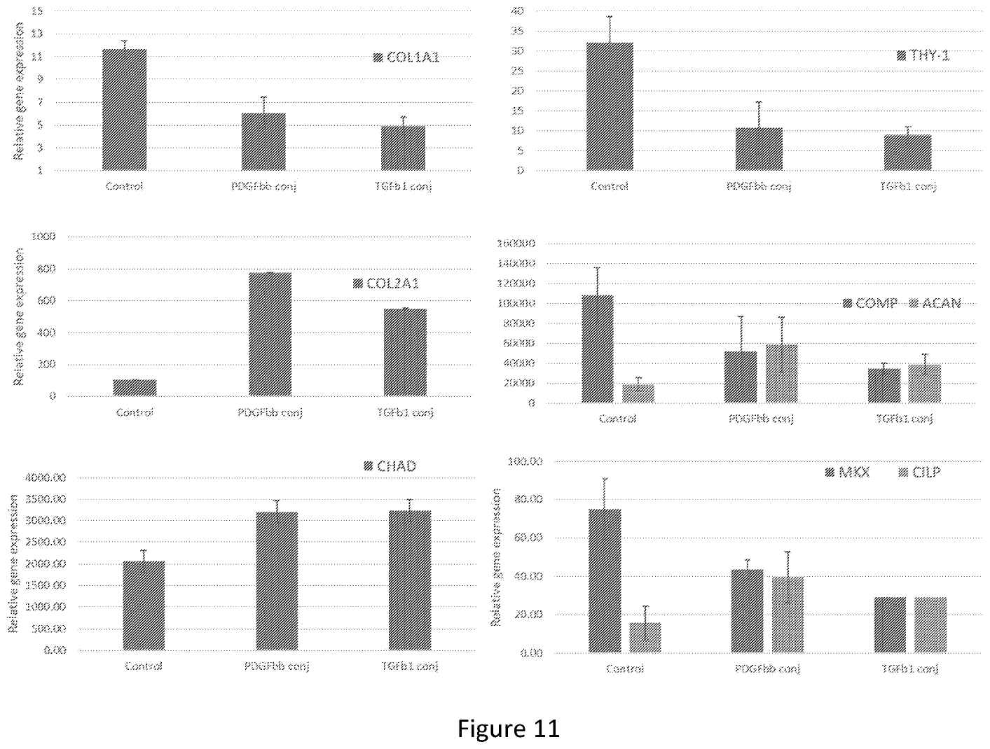

The gene expression profiling also reflected this histology assessment, showing higher COL1A1, THY-1 and MKX in the control condition (FIG. 11) and higher COL2A1, ACAN and CHAD in the growth factor conjugated scaffolds (FIG. 11).

### Example 8: Implantation of ES-MSC Seeded Pneumatospun Collagen Type I Scaffolds into Human Meniscus Ex Vivo Tissues

Human meniscus tissues were obtained following total knee arthroplasty (approved by Scripps Institutional Review Board) and cut sagittal into pieces of approximately 1 cm width. A 4 mm dermal punch was used to create a hole through the meniscus tissue. GA crosslinked pneumatospun collagen type I scaffolds seeded with ES-MSC were implanted into the created defect and cultured in serum-free differentiation medium for 6 weeks.

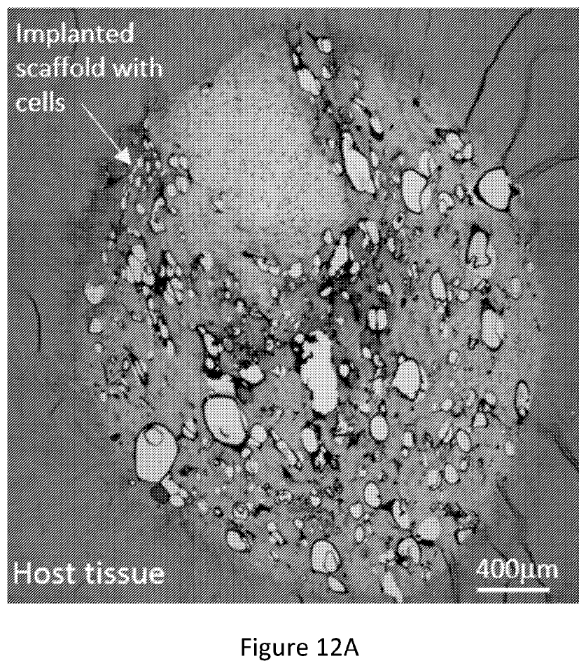

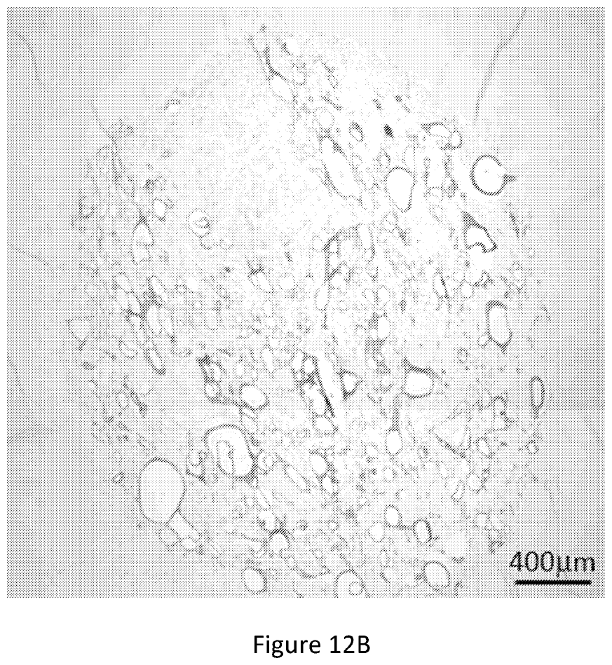

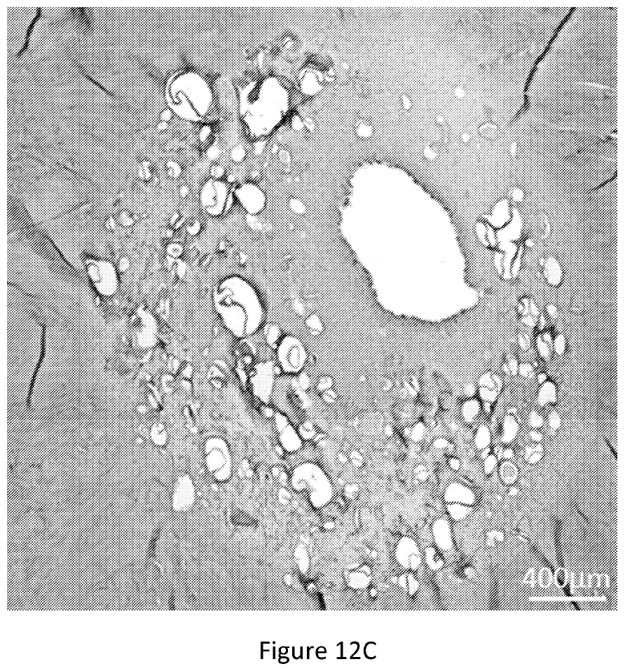

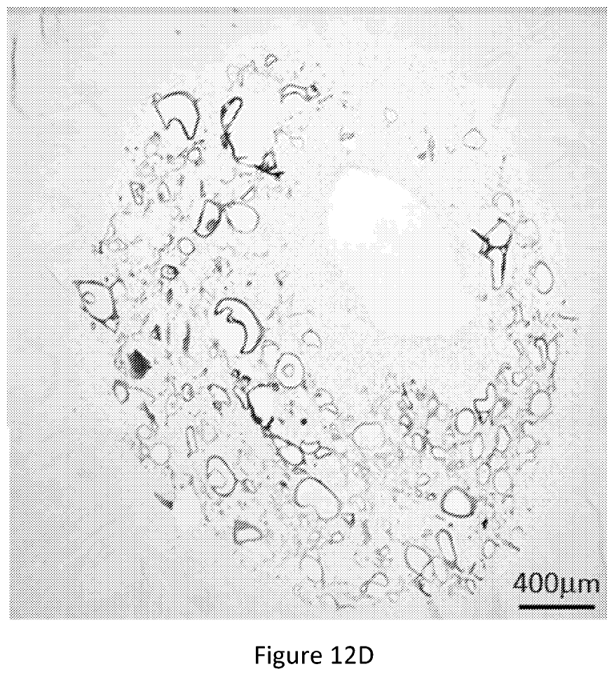

The cell seeded/TGFb1 conjugated scaffolds implanted into OA human tissue defects showed robust neo-tissue regeneration after 6 weeks of culture (FIGS. 12A-12D). The histology staining reveals extensive GAG deposition and positive immunostaining for collagen type I (FIG. 12B) and collagen type II (FIG. 12C).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. A method comprising:
a) applying a gas pressure around a polymer fiber solution comprising a polymer fiber to eject the polymer fiber solution; and
b) collecting the polymer fiber by a collector to form a scaffold,
wherein the scaffold is suitable for use as a meniscal repair device, and
wherein the collector comprises a shape of a meniscus.

2. The method of claim 1, wherein the collector comprises a first curvature and a second curvature on a surface of the collector, wherein a degree of curvature of the first curvature and a degree of curvature of the second curvature are different.

3. The method of claim 1, wherein the scaffold comprises the shape of the meniscus.

4. (canceled)

5. The method of claim 1, wherein the collector comprises a mesh collector or a solid collector.

6. The method of claim 1, wherein the collector rotates at least about 10 revolutions per minute (rpm).

7. (canceled)

8. The method of claim 1, where the collector rotates at a constant speed or at a varying speed.

9. (canceled)

10. The method of claim 1, wherein the collector comprises a rotating collector, a stationary collector, or a translating collector.

11. The method of claim 1, wherein the gas pressure is at least about 1 pound per square inch (psi).

12. (canceled)

13. (canceled)

14. The method of claim 1, wherein the gas pressure comprises air pressure.

15. The method of claim 1, wherein a) comprises applying the gas pressure around the polymer fiber solution using an air brush.

16. The method of claim 15, wherein the polymer fiber solution moves into the air brush via an electromagnetic pressure, an electromechanical pressure, gravity, a hydraulic pressure, a mechanical extrusion pressure, a pneumatic pressure, or a combination thereof.

17. The method of claim 16, wherein the polymer fiber solution moves into the air brush via the mechanical extrusion pressure using a syringe pump.

18. The method of claim 15, wherein the air brush comprises an inner feed line.

19. The method of claim 18, wherein the polymer fiber solution moves inside the inner feed line.

20. The method of claim 19, wherein the polymer fiber solution moves inside the inner feed line continuously during the collecting of b).

21. The method of claim 15, wherein the air brush comprises an outer air line.

22. The method of claim 21, wherein the air moves inside the outer air line.

23. The method of claim 15, wherein the air brush comprises a nozzle to eject the polymer fiber solution.

24. (canceled)

25. The method of claim 23, wherein the nozzle is uniaxial, coaxial, triaxial, tetraaxial, or pentaaxial.

26. The method of claim 23, wherein the nozzle is at least about 30 μm.

27. (canceled)

28. The method of claim 1, wherein the collecting of b) comprises at most about 10 hours.

29. (canceled)

30. (canceled)

31. The method of claim 1, wherein the polymer fiber comprises a type I collagen, a type II collagen, a methacrylated collagen, or a glycosaminoglycan.

32.-48. (canceled)

49. A method comprising:
a) applying a gas pressure around a polymer fiber solution comprising a polymer fiber to eject the polymer fiber solution; and
b) forming a scaffold comprising the polymer,
wherein the scaffold is suitable for use as a meniscal repair device, and
wherein the scaffold has a thickness of at least about 300 micrometer (μm) and a porosity of at least about 40%.

50.-99. (canceled)

100. A method comprising:
a) applying a gas pressure around a polymer fiber solution comprising a plurality of polymer fibers to eject the polymer fiber solution;
b) rotating a collector;
c) collecting at least a subset of the plurality of polymer fibers onto a surface of the collector to form a scaffold; and
d) crosslinking the at least the subset of the plurality of polymer fibers,
wherein the scaffold is suitable for use as a meniscal repair device, and
wherein the collector is rotated on an axis co-linear to an ejection path of a).

101.-158. (canceled)
